(19) United States
(12) Patent Application Publication
(10) Pub. No.: US 2023/0348597 A1
(43) Pub. Date: Nov. 2, 2023

(54) SARS-COV-2 THERAPIES

(71) Applicant: The Jackson Laboratory, Bar Harbor, ME (US)

(72) Inventors: Mikail Dogan, Bar Harbor, ME (US); Derya Unutmaz, Bar Harbor, ME (US)

(73) Assignee: The Jackson Laboratory, Bar Harbor, ME (US)

(21) Appl. No.: 18/182,934

(22) Filed: Mar. 13, 2023

Related U.S. Application Data
(63) Continuation-in-part of application No. PCT/US23/60532, filed on Jan. 12, 2023.
(60) Provisional application No. 63/299,807, filed on Jan. 14, 2022.
(60) Provisional application No. 63/328,713, filed on Apr. 7, 2022.

(51) Int. Cl. C07K 16/28; C07K 14/705; C07K 16/10; A61K 39/00

(52) U.S. Cl. CPC C07K 16/2809; C07K 14/705; C07K 16/1003; A61K 39/4611; A61K 39/4633; C07K 2317/622; C07K 2319/02; C07K 2317/31

(57) ABSTRACT

Disclosed are bispecific molecules combining ACE2 with an anti-CD3 antibody and engineered T cells expressing chimeric antigen receptors that bind to SARS-CoV-2 spike protein, as well as related compositions and methods. The methods and compositions provided can be used for treating early-stage and/or late-stage SARS-CoV-2 infections, independent of the SARS-CoV-2 variant.

### RELATED APPLICATION

This application is a continuation-in-part of international application number PCT/US2023/060532, filed Jan. 12, 2023, which claims the benefit under 35 U.S.C. § 119(e) of U.S. provisional application No. 63/299,807, filed Jan. 14, 2022, and U.S. provisional application No. 63/328,713, filed Apr. 7, 2022, each of which is herein incorporated by reference in its entirety.

### GOVERNMENT LICENSE RIGHTS

This invention was made with government support under U19AI142733-01, awarded by National Institutes of Health. The government has certain rights in the invention.

### REFERENCE TO AN ELECTRONIC SEQUENCE LISTING

The contents of the electronic sequence listing (J022770111U502-SEQ-HJD.xml; Size: 14,822 bytes; and Date of Creation: Mar. 13, 2023) is herein incorporated by reference in its entirety.

### BACKGROUND

Worldwide, Coronavirus disease 19 (COVID-19) caused by severe acute respiratory syndrome coronavirus 2 (SARS-CoV-2) infection has caused close to 5 million deaths and a chronic debilitating condition called Post-Acute COVID-19 Syndrome (PACS) in many millions more. An unprecedented effort by researchers around the world has resulted in the development of a spectrum of preventative and therapeutic approaches at an extraordinary speed. Vaccines focused on virus Spike protein (such as messenger RNA vaccines, non-replicating vector vaccines, and virus-like particle vaccines) are highly efficient in preventing infection. Several therapeutic developments, such as synthetic neutralizing antibodies, monoclonal antibodies to Spike protein and immunomodulators such as corticosteroids, anti-IL-6, anti-IL-1 and Interferon-β-1a agents were shown to have a range of treatment efficacy from non-effective to highly promising. Some of those treatments were repurposed to focus on blocking viral entry while others were used to control the hyperinflammatory immune response during the disease.

Despite advances in antibody treatments and vaccines, COVID-19 caused by SARS-CoV-2 infection remains a major health problem resulting in excessive morbidity and mortality, and the emergence of new variants has reduced the effectiveness of current vaccines.

### SUMMARY

Beyond antibody therapies, specific SARS-CoV-2 immunomodulators have not been developed yet are needed as specific antibodies potentially lose their effectiveness due to new variants. Therefore, the development of treatment approaches that remain effective against SARS-CoV-2 variants is of great interest. The present disclosure provides, in some aspects, bispecific molecules combining angiotensin-converting enzyme 2 (ACE2), a receptor that binds to SARS-CoV-2 Spike protein, with an antibody that specifically binds to a T cell antigen (TA), such as CD3, to target infected cells and the virus. Such molecules are similar to bispecific T-cell engagers in that they bind to a T cell antigen (e.g., CD3) and to a disease antigen (e.g., Spike protein).

The present disclosure also provides, in some aspects, engineered T cells (e.g., primary

CD8 T cells) expressing a chimeric antigen receptor that comprises an extracellular region of ACE2 and thus binds to SARS-CoV-2 Spike protein.

As demonstrated by the data provided herein, both the bispecific ACE2/anti-T cell antigen (TA) molecules when combined with primary T cells and the chimeric antigen receptor-expressing T cells triggered T cell activation and selectively killed Spike protein-expressing targets. Surprisingly, the bispecific molecules comprising ACE2 and an anti-CD3 also neutralized viruses pseudotyped with Spike proteins of SARS-CoV strain, SARS-CoV-2 wild-type and those with mutations derived from variants such as SARS-CoV-2 Alpha, Beta, Delta, and Omicron. Without wishing to be bound by theory, it is thought that these approaches will be effective for current and future Spike protein mutations. Taken together, the approaches reported herein may be used as therapeutic strategies for early- and late-stage COVID-19 infections, independent of the infectious SARS-CoV-2 variant.

Some aspects of the present disclosure provide a polypeptide comprising (a) an antibody that specifically binds to a T cell antigen and (b) a cellular receptor that binds to a coronavirus viral entry protein.

In some embodiments, the T cell antigen is CD3.

In some embodiments, the antibody is selected from an scFv, Fv, F(ab′)2, Fab, and Fab′. In some embodiments, the antibody that specifically binds to a T cell antigen is an scFv.

In some embodiments, the scFv is an anti-CD3 scFv. In some embodiments, the anti-CD3 scFv comprises the amino acid sequence of:

(SEQ ID NO: 1)
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSVEGGSGGSGGSGGSGGVDDIQLTQSPAIMSA
SPGEKVTMTCRASSSVSYMNWYQQKSGTSPKRWIYDTSKVASGVPYRFSG
SGSGTSYSLTISSMEAEDAATYYCQQWSSNPLTFGAGTKLELK.

In some embodiments, the coronavirus viral entry protein is beta coronavirus Spike protein or variant thereof. The beta coronavirus Spike protein may be, for example, a SARS-CoV-2 Spike protein.

In some embodiments, the SARS-CoV-2 Spike protein is a variant SARS-CoV-2 Spike protein. For example, the variant SARS-CoV-2 Spike protein may be selected from Delta (B.1.617.2 and AY lineages), Omicron (B.1.1.529 and BA lineages), Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), 1.617.3, Mu (B.1.621, B.1.621.1), and Zeta (P.2) variants of SARS-CoV-2. Other SARS-CoV-2 variants are contemplated herein.

In some embodiments, the Spike protein is of SARS-CoV strain.

In some embodiments, the Spike protein is of a strain belongs to Coronaviridae family.

In some embodiments, the cellular receptor is angiotensin-converting enzyme 2 (ACE2) receptor. The cellular receptor, in some embodiments, comprises the extracellular domain of human ACE2 receptor.

In some embodiments, the ACE2 extracellular domain comprises the amino acid sequence of:

(SEQ ID NO: 2)
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVS.

In some embodiments, the antibody is linked to the cellular receptor. In some embodiments, the antibody is linked to the cellular receptor through a peptide linker. In some embodiments, the peptide linker comprises the sequence of GGGGS (SEQ ID NO: 3). In some embodiments, the antibody is fused to the cellular receptor.

In some embodiments, the polypeptide further comprises an ACE2 signal peptide. For example, the ACE2 signal peptide may be MSSSSWLLLSLVAVTAA (SEQ ID NO: 4).

In some embodiments, the ACE2 receptor is a modified ACE2 receptor, relative to wild-type ACE2, that does not bind to angiotensin.

In some embodiments, the polynucleotide comprises an amino acid sequence that has at least 80%, at least 85%, at least 90%, at least 95%, or at least 98% identity to the sequence of:

(SEQ ID NO: 5)
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVSGGGGSDIKLQQSGAELARPGASVKMSC
KTSGYTFTRYTMHWVKQRPGQGLEWIGYINPSRGYTNYNQKFKDKATLTT
DKSSSTAYMQLSSLTSEDSAVYYCARYYDDHYCLDYWGQGTTLTVSSVEG
GSGGSGGSGGSGGVDDIQLTQSPAIMSASPGEKVTMTCRASSSVSYMNWY
QQKSGTSPKRWIYDTSKVASGVPYRFSGSGSGTSYSLTISSMEAEDAATY
YCQQWSSNPLTFGAGTKLELK.

In other embodiments, the polypeptide comprises an amino acid sequence that has at least 80%, at least 85%, at least 90%, at least 95%, or at least 98% identity to the sequence of:

(SEQ ID NO: 6)
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADQSIKVRISLKSALGDKAYEWNDNEMYLFRSSVAYA
MRQYFLKVKNQMILFGEEDVRVANLKPRISFNFFVTAPKNVSDIIPRTEV
EKAIRMSRSRINDAFRLNDNSLEFLGIQPTLGPPNQPPVSGGGGSDIKLQ
QSGAELARPGASVKMSCKTSGYTFTRYTMHWVKQRPGQGLEWIGYINPSR
GYTNYNQKFKDKATLTTDKSSSTAYMQLSSLTSEDSAVYYCARYYDDHYC
LDYWGQGTTLTVSSVEGGSGGSGGSGGSGGVDDIQLTQSPAIMSASPGEK
VTMTCRASSSVSYMNWYQQKSGTSPKRWIYDTSKVASGVPYRFSGSGSGT
SYSLTISSMEAEDAATYYCQQWSSNPLTFGAGTKLELK.

Other aspects of the present disclosure provide a polynucleotide encoding the polypeptide of any one of the preceding paragraphs.

Further aspects of the present disclosure provide a polynucleotide encoding an ACE2 signal peptide, an ACE2 extracellular domain, a linker peptide, and an anti-CD3 antibody single-chain variable fragment.

Also provided herein is a polypeptide encoded by a polynucleotide encoding an ACE2 signal peptide, an ACE2 extracellular domain, a linker peptide, and an anti-CD3 antibody single-chain variable fragment.

Some aspects of the present disclosure provide a vector comprising a polynucleotide of any one of the preceding paragraphs (e.g., a polynucleotide encoding an ACE2 signal peptide, an

ACE2 extracellular domain, a linker peptide, and an anti-CD3 antibody single-chain variable fragment). In some embodiments, the vector is a lentiviral vector, retroviral vector, adenoviral vector, adeno-associated viral vector, or herpes simplex viral vector. In some embodiments, the vector is a lentiviral vector.

Other aspects of the present disclosure provide a pharmaceutical composition comprising the polypeptide of any one of the preceding paragraphs (e.g., a polypeptide comprising an ACE2 signal peptide, an ACE2 extracellular domain, a linker peptide, and an anti-CD3 antibody single-chain variable fragment) and a pharmaceutically acceptable excipient.

Some aspects of the present disclosure provide a T cell comprising a chimeric antigen receptor that comprises an extracellular domain of an angiotensin-converting enzyme 2 (ACE2) receptor.

Other aspects of the present disclosure provide a T cell comprising a chimeric antigen receptor that comprises an anti-SARS-CoV-2 Spike scFv.

In some embodiments, the T cell further comprises a CD8 alpha signal peptide, and intracellular 4-1BB co-stimulatory domain, and/or a CD3 (zeta) signaling domain.

Also provided herein are methods comprising administering to a subject the polypeptide of any one of the preceding paragraphs (e.g., a polypeptide comprising an ACE2 signal peptide, an ACE2 extracellular domain, a linker peptide, and an anti-CD3 antibody single-chain variable fragment), a polynucleotide of any one of the preceding paragraphs (e.g., a polynucleotide encoding an ACE2 signal peptide, an ACE2 extracellular domain, a linker peptide, and an anti-CD3 antibody single-chain variable fragment), a vector of any one of the preceding paragraphs, or the pharmaceutical composition. In some embodiments, the subject has a beta coronavirus infection. In some embodiments, the subject has or is at risk of a SARS-CoV-2 infection.

In some embodiments, an antibody is an anti-CD3 scFv comprising the amino acid sequence of SEQ ID NO: 1 or an amino acid sequence having at least 80%, at least 85%, at least 90%, at least 95%, at least 96%, at least 97%, at least 98%, or at least 99% identity to the amino acid sequence of SEQ ID NO: 1.

In some embodiments, a cellular receptor is an angiotensin-converting enzyme 2 (ACE2) receptor. The ACE2 receptor comprises, in some embodiments, the extracellular domain of a wild-type (i.e., naturally occurring isolate) SARS-CoV-2 ACE2 receptor. In some embodiments, the ACE2 extracellular domain comprises the amino acid sequence of SEQ ID NO: 2 or an amino acid sequence having at least 80%, at least 85%, at least 90%, at least 95%, at least 96%, at least 97%, at least 98%, or at least 99% identity to the amino acid sequence of SEQ ID NO: 2.

In some embodiments, an antibody is linked to the cellular receptor. For example, the antibody may be linked to the cellular receptor through a peptide linker. In some embodiments, the peptide linker comprises the amino acid sequence of SEQ ID NO: 3 or an amino acid sequence having at least 80%, at least 85%, at least 90%, at least 95%, at least 96%, at least 97%, at least 98%, or at least 99% identity to the amino acid sequence of SEQ ID NO: 3.

In some embodiments, a polypeptide further comprising an ACE2 signal peptide. In some embodiments, the ACE2 signal peptide comprises the amino acid sequence of SEQ ID NO: 4 or an amino acid sequence having at least 80%, at least 85%, at least 90%, at least 95%, at least 96%, at least 97%, at least 98%, or at least 99% identity to the amino acid sequence of SEQ ID NO: 4.

In some embodiments, an ACE2 receptor is a modified ACE2 receptor, relative to wild-type SARS-CoV-2, that does not bind to angiotensin.

In some embodiments, a polypeptide comprises an amino acid sequence that has at least 80%, at least 85%, at least 90%, at least 95%, or at least 98% identity to the sequence of SEQ ID NO: 5.

In some embodiments, a polypeptide comprises an amino acid sequence that has at least 80%, at least 85%, at least 90%, at least 95%, or at least 98% identity to the sequence of SEQ ID NO: 6.

### BRIEF DESCRIPTION OF THE DRAWINGS

FIGS. 1A-1F show results of engineering human primary CD8 T cells to express 30 chimeric antigen receptor (CAR) molecules targeting SARS-CoV-2 Spike protein expressing cells. FIGS. 1A-1B show an illustration of Spike protein localization on the surface of SARS-CoV-2 infected cells (FIG. 1A) and of full-length SARS-CoV-2 Spike protein mRNA expressing plasmid including the Endoplasmic Reticulum Retention Signal (ERRS) of Spike protein on the C terminal (FIG. 1B). FIG. 1C shows 293 cells transfected with full-length Spike protein (blue histogram) or with VSV-G as a negative control (red histogram) expressing vectors. The cells were stained with ACE2-Fc and anti-Fc-APC secondary antibody, flow cytometry data overlays are shown. FIG. 1D shows 293 cells transduced with a lentivirus encoding a truncated Spike protein gene without the ERRS domain and Green Fluorescent Protein (GFP) as a reporter. Transduced cells were stained with ACE2-Fc and anti-Fc-APC secondary antibody, representative flow cytometry data plots are shown. FIG. 1E shows an illustration of ACE2 CAR and anti-SARS-CoV-2 Spike protein CAR constructs and their expression in CD8 T cells. A constitutive LTR promoter drives ACE2 or anti-Spike CAR and RFP genes separated by an Internal Ribosomal Entry Site (IRES). CAR constructs consist of CD8 alpha signal peptide, ACE2 or single chain variable fragment of an anti-Spike antibody, CD8 Hinge, CD8 transmembrane domain, 4-1BB (CD137) co-stimulatory domain and CD3ζ domain. Lentiviruses containing CARs were used to transduce primary CD8 T cells. FIG. 1F shows the expression of

CAR constructs on CD8 T cells. Activated and transduced CD8 T cells were expanded for 10-12 days and stained with SARS-CoV-2 51 protein fused to mouse Fc, and anti-mouse Fc secondary antibody. Flow cytometry plots showing ACE2 or anti-Spike surface expression versus RFP are shown. Anti-CD19 CAR expressing CD8 T cells were used as control. The experiments were replicated several times with similar results.

FIGS. 2A-2F show the cytotoxic activity of human primary CD8 T cells engineered to express ACE2 CAR or anti-Spike CAR. FIG. 2A shows an illustration of a cytotoxicity assay against Spike-expressing target cells using ACE2 CAR or anti-Spike CAR expressing CD8 T cells as effector cells. FIGS. 2B-2D show CAR-engineered T cell cytotoxicity assays with Spike-expressing 293 target cells at different Effector:Target ratios. CD8 T cells transduced with anti-CD19 CAR lentiviruses were used as control effector cells. Effector CD8 T cells were identified with CD8 staining while target cells were gated based on GFP (Spike) expression. Activation of effector cells and CAR expression and RFP, respectively, were determined with CD25 expression after gating on CD8 T cells. FIG. 2E shows the percent cytotoxicity of ACE2 CAR (blue) and anti-Spike CAR (purple) T cells normalized to anti-CD19 CAR-T cells at different Effector:Target ratios and using Spike-expressing 293 cells as the target. FIG. 2F shows CAR engineered T cells cytotoxicity assays with a Spike-expressing target B cell line (T2 cells). Wild-type CD8 T cells were used as negative control and anti-CD19 CAR expressing CD8 T cells were used as positive control. Panels show representative experiments replicated with similar results.

FIGS. 3A-3F show functional ACE2/anti-CD3 bi-specific T cell engagers against SARS-CoV-2. FIG. 3A shows an illustration describing potential mechanism of action of bispecific ACE2/anti-CD3 recombinant protein. The extracellular domain (ECD) of ACE2 (blue) in bispecific ACE2/anti-CD3 recombinant protein binds to Spike protein (red) expressed on the surface of SARS-CoV-2 infected cells and the anti-CD3 fragment (orange) binds to CD3 molecule (purple) on T cells linking both cell types and inducing the activation of T cells which subsequently results in apoptosis of infected target cells. Bispecific ACE2/anti-CD3 molecule also contains a hemagglutinin (HA) tag at the C terminal. FIG. 3B shows a representation of bispecific ACE2/anti-CD3 molecule and the protein production in 293 cells. A constitutive LTR promoter drives the expression of bispecific ACE2/anti-CD3 molecule and RFP genes separated by an Internal Ribosomal Entry Site (IRES). Bispecific ACE2/anti-CD3 cassette consists of ACE2 signal peptide (SP), ACE2 extracellular domain, a linker peptide, an anti-CD3 antibody single-chain variable fragment, a His-Tag, and a Hemagglutinin (HA) Tag. Lentiviruses expressing bispecific ACE2/anti-CD3 molecules were used to transduce suspension 293 cells that produce and secrete bispecific ACE2/anti-CD3 molecule in their culture supernatant. FIG. 3C shows a representation of the bead-based bispecific ACE2/anti-CD3 molecule capture assay. Fluorescent beads coated with Spike-Receptor binding domain (S-RBD) were used to capture bispecific ACE2/anti-CD3 molecules which were detected via a recombinant CD3-Fc fusion protein and an anti-Fc antibody then subsequently analyzed via flow cytometry. ACE2-Fc molecules were also detected with S-RBD coated beads and anti-Fc antibody. FIG. 3D shows the detection of different concentrations of bispecific ACE2/anti-CD3 molecules (1:10 and 1:300 dilutions were shown in orange and turquoise, respectively) and ACE2-Fc (3 μg/mL) (red) by bead-based bispecific ACE2/anti-CD3 molecule capture assay. Wild-type 293 cell supernatant (Control supe, Blue) and staining buffer (None, Pink) were used as negative controls. FIG. 3E shows binding of bispecific ACE2/anti-CD3 molecule to Spike-GFP expressing T2 cell line and primary human T cells. HA staining of Spike-GFP expressing T2 cells (top) and CD8 T cells (bottom) when combined with bispecific ACE2/anti-CD3 molecule (right plot) or control (Wild-type 293) (left plot) supernatant. FIG. 3F shows that CD25 and GFP expression show activation and cytotoxicity of resting CD8 T cells against Spike/GFP-expressing or control (transduced with GFP-expres sing empty vector) 293 cells in the presence of bispecific ACE2/anti-CD3 molecule or control supernatant. The experiments were replicated with similar results.

FIGS. 4A-4C show the binding of bispecific ACE2/anti-CD3 molecule to Spike proteins with different variant mutations. FIG. 4A shows a representation of bispecific ACE2/anti-CD3 molecule binding to spike protein (wild-type or mutated) expressed on the cell surface membrane and its detection by immunostaining with an anti-HA antibody. Wild-type or mutant Spike proteins (Table 1) were expressed on 293 cells for bispecific ACE2/anti-CD3 molecule/Spike binding assay. FIG. 4B shows geometric mean intensity of anti-HA antibody staining used to detect bispecific ACE2/anti-CD3 molecules on mutant Spike-expressing cells. Unpaired t test was used to determine the statistical significance. FIG. 4C shows CD25 expression of CD8 T cells indicating their activation when co-cultured with wild-type and mutated Spike protein plasmid transfected 293 cells (used in FIG. 4B) in the presence of bispecific ACE2/anti-CD3 molecule supernatant.

FIGS. 5A-5C show binding of bispecific ACE2/anti-CD3 molecule to SARS-CoV-2 Spike protein variants on pseudotyped lentiviruses for virus neutralization. FIG. 5A shows a schematic illustration of virus neutralization assay. bispecific ACE2/anti-CD3 molecule and Spike (wild-type and mutated) pseudotyped lentiviruses are pre-incubated then added to ACE2-overexpressing 293 cells. FIG. 5B shows representative FACS plots show neutralization data of the delta variant pseudotyped virus infection when pre-incubated with different concentrations of ACE2-Fc (top panel) or different dilutions of bispecific ACE2/anti-CD3 molecule supernatant (bottom panel). The infection levels were determined 3 days later via flow cytometry based on GFP expression. FIG. 5C shows a line graph representing virus neutralization data of the lentiviruses pseudotyped with different Spike protein variants when pre-incubated with bispecific ACE2/anti-CD3 molecule at different bispecific ACE2/anti-CD3 molecule:virus ratios then added to ACE2-overexpressing 293s. These experiments were replicated twice with similar results.

FIG. 6 shows elective cytotoxicity of ACE2 CAR and anti-Spike CAR-T cells for spike expressing target cells. CAR engineered T cells cytotoxicity assays with Spike expressing and control 293 target cells. Control 293s were engineered with a GFP-expressing empty vector. Spike-expressing and control 293s were identified with GFP expression. Effector cells were identified by CD8 staining. T cell activation was determined via CD25 staining. CAR expressing T cells co-expressed RFP with CAR constructs.

FIG. 7 shows the determination of bispecific ACE2/anti-CD3 molecule concentrations by a capture assay. Area under the curve (AUC) values of bispecific ACE2/anti-CD3 molecules in supernatants from different conditions. Supernatants from molecule secreting and wild-type suspension 293 cells were collected at several timepoints representing different cell densities ranging from 3 to 7 million/mL. bispecific ACE2/anti-CD3 molecules taken from 3 million/mL cell culture supernatant were concentrated 5-folds and 30-folds. Flow through supernatant from the concentration process (Filter flow through) and wild-type control supernatant were used as controls. Fluorescent beads coated with Spike-Receptor binding domain (S-RBD) were used to capture bispecific ACE2/anti-CD3 molecules in supernatants titrated from 1:1 to 1:1000 by 10-fold serial dilutions were detected via a recombinant CD3-Fc fusion protein and an anti-Fc antibody. Geometric mean intensity of anti-Fc antibody fluorescence was used to generate curves which were used to calculate the area under the curve values.

FIGS. 8A-8E show the binding of ACE2 bispecific T-cell engager to wild-type and Omicron variant Spike proteins. FIG. 8A shows the results of a bead-based bispecific ACE2/anti-CD3/Spike binding assay. Geometric mean values of anti-Fc antibody fluorescence in flow cytometry were used to quantify the fluorescent intensity of samples. FIG. 8B shows the area under the curve (AUC) values of bead-based bispecific ACE2/anti-CD3/Spike binding assay data from FIG. 8A. Experiments were replicated three times. FIG. 8C is an illustration of bispecific ACE2/anti-CD3 binding to spike protein (wild-type or Omicron) expressed on the cell surface, and its detection by immunostaining with an anti-HA antibody. FIG. 8D shows a bispecific ACE2/anti-CD3 binding assay on wild-type and Omicron Spike-expressing 293 cells. Geometric mean intensity of anti-HA antibody staining used to quantify the affinity of bispecific ACE2/anti-CD3 molecules on Spike-expressing cells. FIG. 8E shows the area under the curve (AUC) values of bead-based bispecific ACE2/anti-CD3/Spike binding assay data from FIG. 8D. The experiments were replicated twice with similar results.

FIG. 9 shows the neutralization of lentiviruses pseudotyped with SARS-CoV, SARS-CoV-2 wild-type, Delta and Omicron Spike proteins by bispecific ACE2/anti-CD3. The line graph represents virus neutralization data of the lentiviruses pseudotyped with different Spike proteins that were pre-incubated with bispecific ACE2/anti-CD3 at different bispecific ACE2/anti-CD3:virus ratios then added to ACE2-overexpressing 293 cells. The experiments were replicated twice with similar results.

### DETAILED DESCRIPTION

Despite advances in vaccine development, COVID-19 is still a major cause of morbidity and mortality in the USA and throughout the world. Rapid evolution of the virus is also a major concern, suggesting the need to develop novel effective treatment strategies, as SARS-CoV-2 specific targeted therapeutic approaches such as monoclonal antibody therapies can lose their effectiveness as new escape variants emerge. To mitigate or override these potential problems, the present disclosure provides, in some embodiments, synthetic biology approaches to develop synthetic molecules that can bridge T cells with SARS-CoV-2 infected cells, through recognition of cell surface expression of virus Spike protein and eliminate them through cytotoxic activity.

SARS-CoV-2 uses its Spike protein to bind to the key host receptor Angiotensin-Converting Enzyme 2 (ACE2) on target cell surface for cell entry and mutations in Spike protein result in higher affinity of the virus to ACE2 and/or a better escaping mechanism from the immune system. Following the cell entry, SARS-CoV-2 generates viral components by taking over the protein synthesis machinery of the host cell and displays Spike protein on the cell membrane. Using ACE2 molecule to target these Spike-expressing infected cells may be an effective strategy in preventative and therapeutic approaches to COVID-19 because ACE2 receptor is compatible to the binding of forthcoming mutant Spike proteins. An example of a SARS-CoV-2 Spike protein sequence is:

(SEQ ID NO: 7)
MFVFLVLLPL VSSQCVNLTT RTQLPPAYTN SFTRGVYYPD
KVFRSSVLHS TQDLFLPFFS NVTWFHAIHV SGTNGTKRFD
NPVLPFNDGV YFASTEKSNI IRGWIFGTTL DSKTQSLLIV
NNATNVVIKV CEFQFCNDPF LGVYYHKNNK SWMESEFRVY
SSANNCTFEY VSQPFLMDLE GKQGNFKNLR EFVFKNIDGY
FKIYSKHTPI NLVRDLPQGF SALEPLVDLP IGINITRFQT
LLALHRSYLT PGDSSSGWTA GAAAYYVGYL QPRTFLLKYN
ENGTITDAVD CALDPLSETK CTLKSFTVEK GIYQTSNERV
QPTESIVRFP NITNLCPFGE VFNATRFASV YAWNRKRISN
CVADYSVLYN SASFSTFKCY GVSPTKLNDL CFTNVYADSF
VIRGDEVRQI APGQTGKIAD YNYKLPDDFT GCVIAWNSNN
LDSKVGGNYN YLYRLFRKSN LKPFERDIST EIYQAGSTPC
NGVEGFNCYF PLQSYGFQPT NGVGYQPYRV VVLSFELLHA
PATVCGPKKS TNLVKNKCVN FNFNGLIGTG VLTESNKKFL
PFQQFGRDIA DTTDAVRDPQ TLEILDITPC SFGGVSVITP
GTNTSNQVAV LYQDVNCTEV PVAIHADQLT PTWRVYSTGS
NVFQTRAGCL IGAEHVNNSY ECDIPIGAGI CASYQTQTNS
PRRARSVASQ SIIAYTMSLG AENSVAYSNN SIAIPTNFTI
SVTTEILPVS MTKTSVDCTM YICGDSTECS NLLLQYGSFC
TQLNRALTGI AVEQDKNTQE VFAQVKQIYK TPPIKDFGGF
NFSQILPDPS KPSKRSFIED LLFNKVTLAD AGFIKQYGDC
LGDIAARDLI CAQKFNGLTV LPPLLTDEMI AQYTSALLAG
TITSGWTFGA GAALQIPFAM QMAYRFNGIG VTQNVLYENQ
KLIANQFNSA IGKIQDSLSS TASALGKLQD VVNQNAQALN
TLVKQLSSNF GAISSVLNDI LSRLDKVEAE VQIDRLITGR
LQSLQTYVTQ QLIRAAEIRA SANLAATKMS ECVLGQSKRV
DFCGKGYHLM SFPQSAPHGV VFLHVTYVPA QEKNFTTAPA
ICHDGKAHFP REGVFVSNGT HWFVTQRNFY EPQIITTDNT
FVSGNCDVVI GIVNNTVYDP LQPELDSFKE ELDKYFKNHT
SPDVDLGDIS GINASVVNIQ KEIDRLNEVA KNLNESLIDL
QELGKYEQYI KWPWYIWLGF IAGLIAIVMV TIMLCCMTSC
CSCLKGCCSC GSCCKFDEDD SEPVLKGVKL HYT

Provided herein are polypeptides comprising an antibody that specifically binds to a T cell antigen and a cellular receptor that binds to a coronavirus viral entry protein (e.g., a SARS-CoV-2 Spike protein, such as the Spike protein shown as SEQ ID NO: 7). In some embodiments, the disclosure provides bispecific molecules combining ACE2 with an anti-TA antibody (e.g., an anti-CD3 antibody) to target infected cells and the virus. The present disclosure also provides, in some aspects, an engineered T cell expressing an anti-SARS-CoV-2 Spike protein antibody (e.g., scFv) or a CAR that comprises an extracellular region of ACE2, which binds to SARS-CoV-2 Spike protein.

### I. Therapeutic Agents

The present disclosure provides, in some embodiments, a polypeptide comprising (a) an antibody that specifically binds to a T cell antigen and (b) a cellular receptor that binds to a coronavirus viral entry protein (e.g., a Spike protein). In some embodiments, the polypeptide comprises an engineered, bispecific ACE2/anti-CD3 molecule to target both coronavirus-infected cells and the coronavirus (e.g., the SARS-CoV-2 virus). Other aspects of the disclosure provide T cells comprising chimeric antigen receptors (CARs) that comprise an extracellular domain of a ACE2 or an anti-coronavirus Spike protein antibody (e.g., an anti-SARS-CoV-2 Spike protein scFv).

### 1. Bispecific Molecules

In some embodiments, the polypeptide is a bispecific ACE2/anti-TA molecule (e.g., a bispecific ACE2/anti-CD3 molecule). In some embodiments, the bispecific ACE2/anti-TA molecule is engineered to bind to CD3 on T cells via an anti-CD3 single chain variable fragment (scFv) and to target a SARS-CoV-2-infected cell via ACE2, a receptor that binds to SARS-CoV-2 Spike protein. Upon bridging the T cells with a target cell, the bispecific molecules trigger T cell activation and subsequent target cell cytotoxicity. In some embodiments, the antibody (e.g., anti-CD3) is linked to the cellular receptor (e.g., extracellular domain of ACE2), for example, with a peptide linker, as described elsewhere herein. Exemplary bispecific ACE2/anti-CD3 amino acid sequences are provided as SEQ ID NO: 5 and SEQ ID NO: 6. Additional aspects of the disclosure provide polynucleotides encoding the bispecific molecules, as well as vectors comprising the polynucleotides, as is described herein.

The data provided herein shows that that bispecific ACE2/anti-CD3 molecules lead to T cell activation in the presence of Spike expressing targets, and mediate cytotoxicity to these targets. In addition, bispecific ACE2/anti-CD3 molecules act as a decoy receptor for wild type and mutated Spike pseudotyped viruses and neutralize them regardless of their mutations. Taken together, these results suggest that the bispecific ACE2/anti-CD3 molecules described herein may be used to redirect cytotoxic immune cells towards SARS-CoV-2 infected host cells and to neutralize variant strains of the virus.

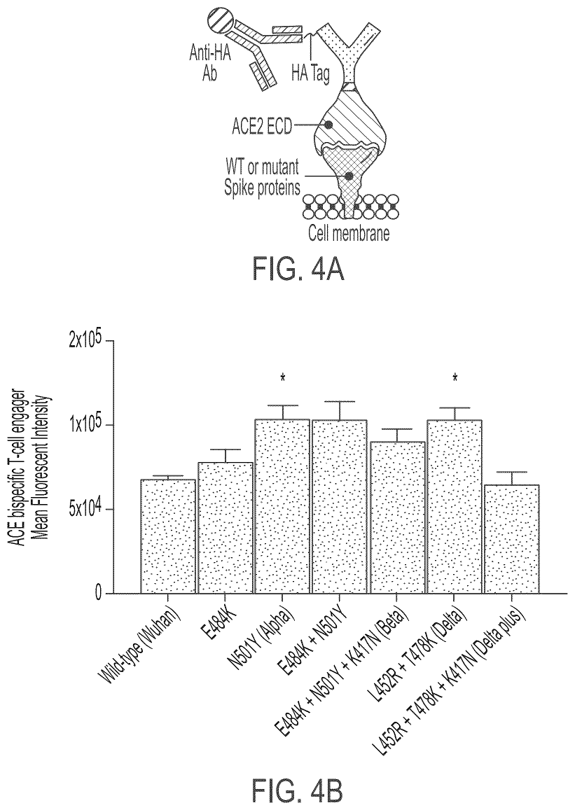

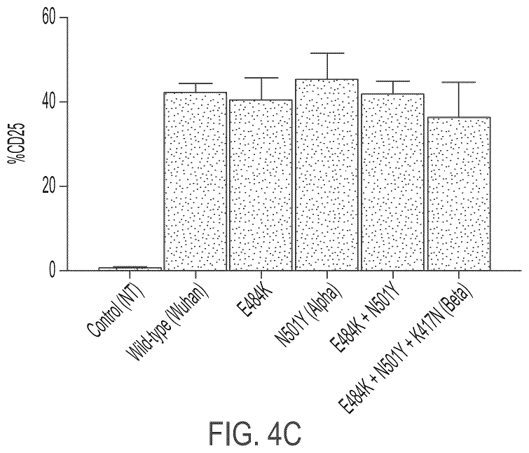

As discussed above, the data provided herein shows that bispecific ACE2/anti-CD3 molecule that trigger effective CD8 T cell activation, resulting in selective killing of Spike-positive cells. Compared to current treatments (such as neutralizing antibodies or anti-viral therapeutics), the bispecific ACE2/anti-CD3 molecule approach may be effective both at early stages (as a neutralizer of the virus entry) and later stages of the infection, when antibody immune defenses are breached, and T cells become more important in restricting the spread of the virus in vivo. A major advantage of the bispecific ACE2/anti-CD3 molecule approach is using ACE2, the key host receptor of SARS-CoV-2, as the Spike protein recognizing part of the bi-specific antibody. As shown in FIGS. 4A-4C, mutated Spike proteins from variants of concern could be targeted, regardless of their mutations.

In addition to recognizing mutated Spike proteins, it was found that the ACE2 component of the bispecific ACE2/anti-CD3 molecule functioned as a decoy receptor and neutralized the virus, preventing it from infecting the cells. Therefore, the bispecific ACE2/anti-CD3 molecule could be used prophylactically, as the neutralization feature of the bispecific ACE2/anti-CD3 molecule would conceivably have synergistic effect with the cytotoxic effect by engaging T cells towards infected cells. Considering the immunity of ACE2 to the Spike mutations, both as a CD3 T cell redirecting molecule and a decoy receptor, the efficacy of the bispecific ACE2/anti-TA molecule treatment is unlikely to be diminished by variants arising during COVID-19 pandemic or in possible future coronavirus (e.g., SARS) pandemics.

As presented herein, bispecific ACE2/anti-CD3 molecules could be used to target SARS-CoV-2 infected host cells and the virus itself, and may be alternative future therapeutic strategies for COVID-19.

### a. Anti-T Cell Antigen (TA) Antibodies

An antibody is an immunoglobulin molecule that recognizes and specifically binds a target, such as a protein, polypeptide, peptide, carbohydrate, polynucleotide, lipid, or a combination of any of the foregoing, through at least one antigen-binding site wherein the antigen-binding site is usually within the variable region of the immunoglobulin molecule. As used herein, the term “antibody” encompasses intact (whole) polyclonal antibodies, intact monoclonal antibodies, antibody fragments (such as Fab, Fab′, F(ab′)2, and Fv fragments), single chain Fv (scFv) antibodies, multispecific antibodies, bispecific molecules, monospecific antibodies, monovalent antibodies, chimeric antibodies, humanized antibodies, human antibodies, fusion proteins comprising an antigen-binding site of an antibody, antibody mimetics, and any other modified immunoglobulin molecule comprising an antigen-binding site as long as the antibodies exhibit the desired biological activity. In some embodiments, the antibody is selected from an antibody fragment, such as scFv, Fv, F(ab')2, Fab, Fab', and Fv. In some embodiments, the antibody is an scFv (single-chain fragment variable). An scFv is a fusion protein comprising the variable regions of the heavy (VH) and light (VL) chains of immunoglobulins.

The antibody, in some embodiments, specifically binds to a T cell antigen (e.g., the epitope of a T cell antigen). An epitope is the portion of an antigen (e.g., a T cell antigen) capable of being recognized and specifically bound by a particular antibody. An epitope typically includes at least 3, and more usually, at least 5, 6, 7, or 8-10 amino acids in a unique spatial conformation. “Specifically binds to” or is “specific for” refers to measurable and reproducible interactions such as binding between a target (e.g., a T cell antigen) and antibody, which is determinative of the presence of the target in the presence of a heterogeneous population of molecules including biological molecules. For example, an antibody that specifically binds to a specific T cell antigen is an antibody that binds the T cell antigen with greater affinity, more readily, and/or with greater duration than it binds to other targets. In some embodiments, the T cell antigen is CD3, CDS, or CD7. In some embodiments, the T cell antigen is CD3. CD3, cluster of differentiation 3, is a multimeric protein complex comprising four polypeptide chains: epsilon (ε), gamma (γ), delta (δ) and zeta (ζ), that assemble and function as three pairs of dimers (εγ, εδ, ζζ). The molecule plays a role in antigen recognition, T cell activation, and triggers antigen-specific immune responses.

In some embodiments, the scFv is an anti-CD3 scFv. In some embodiments, the anti-CD3 scFv comprises the following amino acid sequence:

(SEQ ID NO: 1)
DIKLQQSGAELARPGASVKMSCKTSGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSSVEGGSGGSGGSGGSGGVDDIQLTQSPAIMSA
SPGEKVTMTCRASSSVSYMNWYQQKSGTSPKRWIYDTSKVASGVPYRFSG
SGSGTSYSLTISSMEAEDAATYYCQQWSSNPLTFGAGTKLELK.

### b. Cellular Receptors Binding Coronavirus Viral Entry Proteins

The polypeptide, in some embodiments, comprises a cellular receptor that binds to a coronavirus viral entry protein. Human coronaviruses are highly contagious, enveloped, positive sense single-stranded RNA viruses of the Coronaviridae family. Two sub-families of Coronaviridae are known to cause human disease. In some embodiments, the coronavirus is a β-coronavirus (betacoronavirus). In some embodiments, the betacoronavirus is selected from the group consisting of MERS-CoV, SARS-CoV, SARS-CoV-2, HCoV-OC43, HCoV-229E, HCoV-NL63, HCoV-NL, HCoV-NH and HCoV-HKU1. The genome of coronaviruses includes a variable number of open reading frames that encode accessory proteins, nonstructural proteins, and structural proteins. Most of the antigenic peptides are located in the structural proteins. Spike surface glycoprotein (S), a small envelope protein (E), matrix protein (M), and nucleocapsid protein (N) are four main structural proteins. In some embodiments, the coronavirus viral entry protein is a Spike protein, as Spike proteins contribute to cell tropism and virus entry and are capable of inducing neutralizing antibodies (NAb) and protective immunity.

As used herein, the term “Spike protein” refers to a glycoprotein that forms homotrimers protruding from the envelope (viral surface) of viruses including betacoronaviruses. Trimerized Spike protein facilitates entry of the virion into a host cell by binding to a receptor on the surface of a host cell (e.g., the ACE2 receptor) followed by fusion of the viral and host cell membranes. The S protein is a highly glycosylated and large type I transmembrane fusion protein that is made up of 1,160 to 1,400 amino acids, depending upon the type of virus. Betacoronavirus Spike proteins comprise between about 1100 to 1500 amino acids.

In some embodiments, the polypeptide comprises a cellular receptor that binds to a coronavirus Spike protein or a variant thereof. As used herein, a “variant” refers to a molecule that differs in its amino acid sequence from wild-type (naturally-occurring), native, or a reference protein sequence. As an example, a variant may comprise substitutions, deletions, and/or insertions at certain positions within the amino acid sequence, as compared to a native or reference sequence. In some embodiments, variants possess at least 50% identity to a wild-type, native or reference sequence. In some embodiments, variants share at least 80%, or at least 90% identity with a wild-type, native, or reference sequence.

In some embodiments, the coronavirus Spike protein is a SARS-CoV-2 Spike protein (e.g., SEQ ID NO: 7). In some embodiments, the Spike protein is a variant SARS-CoV-2 protein. Examples of variant SARS-CoV-2 strains include, but are not limited to, Delta (B.1.617.2 and AY lineages), Omicron (B.1.1.529 and BA lineages), Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), 1.617.3, Mu (B.1.621, B.1.621.1), and Zeta (P.2). In some embodiments, the Spike protein is a SARS-CoV-2 Omicron Spike protein.

As noted above, a cellular receptor that binds coronavirus viral entry proteins is angiotensin-converting enzyme 2 (ACE2). Therefore, in some embodiments, the polypeptide comprises ACE2 (e.g., wild-type human ACE2). Full-length human ACE2 is 805 amino acids in length, of which amino acids 1-17 is a signal peptide that is cleaved from the mature protein. See NCBI Reference Sequence NP 001358344.1; see also UniProtKB Reference Q9BYF1. The ACE2 extracellular domain comprises an N-terminal peptidase domain (aa 18-614) and a C-terminal dimerization domain, also referred to as a “collectrin” domain (aa 615-740). Recent studies have revealed the structural basis of the high-affinity ACE2-spike interaction through the Spike protein's receptor binding domain (RBD) and note that the ACE2-RBD co-structure shows a large, flat binding interface primarily comprising the N-terminal helices of ACE2 (residues 18-90), with secondary interaction sites spanning residues 324-361. Therefore, in some embodiments, the cellular receptor comprises the extracellular domain of the wild-type ACE2. In some embodiments, the extracellular domain of ACE2 comprises the following amino acid sequence:

(SEQ ID NO: 2)
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVS.

In some embodiments, the polypeptide further comprises an ACE2 signal peptide, such as the following amino acid sequence: MSSSSWLLLSLVAVTAA (SEQ ID NO: 4).

In some embodiments, the ACE2 is modified ACE2. As used herein, “modified” refers to any modification of a natural or unnatural amino acid relative to wild-type ACE2. For example, in some embodiments, the ACE2 has been modified such that it does not bind to angiotensin, or it binds to angiotensin with a lower affinity than wild-type ACE2.

### 2. Coronavirus Spike Protein-Specific CAR T Cells

The present disclosure also provides, in some embodiments, a synthetic biology approach to engineer primary human CD8 T cells to express Spike protein-specific chimeric antigen receptors (ACE2 CAR or anti-Spike CAR) with ACE2 or anti-Spike antibody on the extracellular domain to target coronavirus-infected cells (e.g., SARS-CoV-2-infected cells). The ACE2-CAR and anti-Spike CAR-expressing CD8 T cells become activated and selectively kill different types of target cells expressing Spike protein (e.g., SARS-CoV-2 Spike protein) on their surface. Taken together, these results suggest that the chimeric antigen receptors described herein may be used to redirect cytotoxic immune cells towards SARS-CoV-2 infected host cells.

CD8 T cells expressing chimeric antigen receptors (CARs) specific to Spike protein with an anti-Spike antibody or ACE2 surface domain on the extracellular region were generated and their effectiveness against different cell types expressing Spike proteins was tested. As presented herein, engineered CAR-T cells (anti-Spike CARs and ACE2 CARs) became activated and killed the Spike-expressing target cells selectively. Although cancer cells have predominantly been the focus of adaptive cellular immunotherapies, studies have suggested that autoimmune and infectious diseases could also be targeted via such approaches. As presented herein, engineered CD8 T cells expressing Spike protein-specific chimeric antigen receptors could be used to target SARS-CoV-2 infected host cells and the virus itself, and may be alternative future therapeutic strategies for COVID-19.

As used herein, the terms “CAR T” or “CAR-T cells” refer to a T-cell or population thereof, which has been modified through methods to express a chimeric antigen receptor (CAR) on the T-cell surface. The CAR is a polypeptide having a pre-defined binding specificity to a desired target expressed operably connected to (e.g., as a fusion, separate chains linked by one or more disulfide bonds, etc.) the intracellular part of a T-cell activation domain. The CAR T cell may be derived from any human T cell. For instance, a CAR T cell may be derived from a human T cell that has been isolated from a human donor, e.g., a human donor that is not the subject treated according to the method (i.e., the CAR T cells are allogeneic), but instead a healthy human donor. CAR T cells, and cells derived therefrom, may be derived from, for example, isolated T cells that have not been passaged in culture, or T cells that have been passaged and maintained under cell culture conditions without immortalization. In some embodiments, the CAR T cells are CD8+ T cells, such as cytotoxic T cells. In some embodiments, the CAR T cells are CD4+ T cells, such as T helper cells.

In some embodiments, the CAR T cells comprise an extracellular domain of ACE2 or a modified ACE2, as described herein. In some embodiments, the CAR T cells comprise an anti-coronavirus Spike protein scFv (e.g., an anti-SARS-CoV-2 Spike protein scFv) described herein.

In some embodiments, a CAR of the present disclosure will comprise at least an extracellular domain, a transmembrane domain, and an intracellular domain. In some embodiments, the extracellular domain comprises a target-specific binding element otherwise referred to as an extracellular ligand-binding domain or moiety (e.g., ACE2 or an anti-SARS-CoV-2 Spike protein scFv).

In some embodiments, the intracellular domain, or cytoplasmic domain, comprises at least one co-stimulatory domain and one or more signaling domains.

In some embodiments, a CAR comprises a transmembrane domain which links the extracellular ligand-binding domain with the intracellular signaling and co-stimulatory domains via a hinge region or spacer sequence. Hinge regions may be derived from all or part of naturally occurring molecules, such as from all or part of the extracellular region of CD8, CD4 or CD28, or from all or part of an antibody constant region. In some embodiments, a hinge domain (transmembrane domain) can comprise a part of a human CD8 alpha signal peptide.

In some embodiments, the intracellular domain, or cytoplasmic domain, comprises at least one co-stimulatory domain and one or more signaling domains. Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular stimulatory domain can include one or more cytoplasmic signaling domains that transmit an activation signal to the T cell following antigen binding. Such cytoplasmic signaling domains can include, without limitation, a CD3 zeta signaling domain. The intracellular stimulatory domain can also include one or more intracellular co-stimulatory domains that transmit a proliferative and/or cell-survival signal after ligand binding. In some cases, the co-stimulatory domain can comprise one or more of the following costimulatory domains: 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, or any combination thereof. In some embodiments, the co-stimulatory domain is a 4-1BB co-stimulatory domain.

In some embodiments, the CAR T cells further comprise a leader sequence (e.g., a CD8 alpha signal peptide), an intracellular co-stimulatory domain (e.g., a 4-1BB (CD137) co-stimulatory domain), and/or a signaling domain (e.g., a CD3 (zeta) signaling domain).

As presented herein, engineered CD8 T cells expressing Spike protein-specific chimeric antigen receptors could be used to target SARS-CoV-2 infected host cells and the virus itself, and may be alternative future therapeutic strategies for COVID-19.

### II. Polypeptides

The present disclosure provides, as described above, a polypeptide comprising an antibody that specifically binds to a T cell antigen and a cellular receptor that binds to a coronavirus viral entry protein (e.g., a Spike protein).

In some embodiments, the antibody (e.g., anti-CD3) is linked to the cellular receptor (e.g., extracellular domain of ACE2). In some embodiments, the antibody and cellular receptor are directed linked (e.g., without a spacer or linker). In some embodiments, the antibody and the cellular receptor are linked via a peptide linker. As used herein, the term “linker” refers to a linker amino acid sequence inserted between a first polypeptide (e.g., the antibody) and a second polypeptide (e.g., the cellular receptor). Linkers do not adversely affect the expression, secretion, or bioactivity of the polypeptide, nor do they elicit an immune response. In some embodiments, the linker is a rigid linker or a flexible linker.

In some embodiments, the linker can be characterized as flexible. Flexible linkers are usually applied when the joined domains require a certain degree of movement or interaction. They are generally composed of small, non-polar (e.g., Gly) or polar (e.g., Ser or Thr) amino acids. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. The most commonly used flexible linkers have sequences consisting primarily of stretches of Gly and Ser residues (“GS” linker). In some embodiments, the linker is flexible. In some embodiments, the linker comprises GGGGS (SEQ ID NO: 3).

In some embodiments, a bispecific ACE2/anti-CD3 molecule of the disclosure comprises the following amino acid sequence:

(SEQ ID NO: 5)
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVSGGGGSDIKLQQSGAELARPGASVKMSC
KTSGYTFTRYTMHWVKQRPGQGLEWIGYINPSRGYTNYNQKFKDKATLTT
DKSSSTAYMQLSSLTSEDSAVYYCARYYDDHYCLDYWGQGTTLTVSSVEG
GSGGSGGSGGSGGVDDIQLTQSPAIMSASPGEKVTMTCRASSSVSYMNWY
QQKSGTSPKRWIYDTSKVASGVPYRFSGSGSGTSYSLTISSMEAEDAATY
YCQQWSSNPLTFGAGTKLELK.

In some embodiments, the polypeptide comprises an amino acid sequence that has at least 80% identity to SEQ ID NO: 5. In some embodiments, the polypeptide comprises an amino acid sequence that has at least 85% identity to SEQ ID NO: 5. In some embodiments, the polypeptide comprises an amino acid sequence that has at least 90% identity to SEQ ID NO: 5. In some embodiments, the polypeptide comprises an amino acid sequence that has at least 95% identity to SEQ ID NO: 5. In some embodiments, the polypeptide comprises an amino acid sequence that has at least 96% identity to SEQ ID NO: 5. In some embodiments, the polypeptide comprises an amino acid sequence that has at least 97% identity to SEQ ID NO: 5. In some embodiments, the polypeptide comprises an amino acid sequence that has at least 98% identity to SEQ ID NO: 5.

In some embodiments, the polypeptide comprises an amino acid sequence that has at least 99% identity to SEQ ID NO: 5.

In some embodiments, a bispecific ACE2/anti-CD3 molecule of the disclosure comprises the following amino acid sequence:

(SEQ ID NO: 6)
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADQSIKVRISLKSALGDKAYEWNDNEMYLFRSSVAYA
MRQYFLKVKNQMILFGEEDVRVANLKPRISFNFFVTAPKNVSDIIPRTEV
EKAIRMSRSRINDAFRLNDNSLEFLGIQPTLGPPNQPPVSGGGGSDIKLQ
QSGAELARPGASVKMSCKTSGYTFTRYTMHWVKQRPGQGLEWIGYINPSR
GYTNYNQKFKDKATLTTDKSSSTAYMQLSSLTSEDSAVYYCARYYDDHYC
LDYWGQGTTLTVSSVEGGSGGSGGSGGSGGVDDIQLTQSPAIMSASPGEK
VTMTCRASSSVSYMNWYQQKSGTSPKRWIYDTSKVASGVPYRFSGSGSGT
SYSLTISSMEAEDAATYYCQQWSSNPLTFGAGTKLELK.

In some embodiments, the polypeptide comprises an amino acid sequence that has at least 80% identity to SEQ ID NO: 6. In some embodiments, the polypeptide comprises an amino acid sequence that has at least 85% identity to SEQ ID NO: 6. In some embodiments, the polypeptide comprises an amino acid sequence that has at least 90% identity to SEQ ID NO: 6. In some embodiments, the polypeptide comprises an amino acid sequence that has at least 95% identity to SEQ ID NO: 6. In some embodiments, the polypeptide comprises an amino acid sequence that has at least 96% identity to SEQ ID NO:6. In some embodiments, the polypeptide comprises an amino acid sequence that has at least 97% identity to SEQ ID NO: 6. In some embodiments, the polypeptide comprises an amino acid sequence that has at least 98% identity to SEQ ID NO: 6. In some embodiments, the polypeptide comprises an amino acid sequence that has at least 99% identity to SEQ ID NO: 6.

As used herein, the term “identity” refers to a relationship between the sequences of two or more polypeptides or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related polypeptides or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. It is understood that identity depends on a calculation of percent identity but may differ in value due to gaps and penalties introduced in the calculation. Such tools for alignment include those of the BLAST suite (Stephen F. Altschul, et al (1997), “Gapped BLAST and PSI-BLAST: a new generation of protein database search programs”, Nucleic Acids Res. 25:3389-3402).

### III. Polynucleotides

The disclosure, in some aspects, provides polynucleotides encoding any one of the polypeptides described herein. A polynucleotide (also referred to herein as a nucleic acid or a nucleic acid molecule) is a polymer of nucleotides of any length and may comprise DNA, RNA (e.g., messenger RNA (mRNA)), or a combination of DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide encoding a polypeptide refers to the order or sequence of nucleotides along a strand of deoxyribonucleic acid deoxyribonucleotides. The order of these deoxyribonucleotides determines the order of amino acids along the polypeptide (e.g., protein) chain. Thus, a nucleic acid sequence encodes the amino acid sequence. When used in reference to nucleotide sequences, a “sequence” may comprise DNA and/or RNA (e.g., messenger RNA) and may be single and/or double stranded. Nucleic acid sequences may be modified, e.g., mutated, relative to naturally occurring nucleic acid sequences, for example.

The disclosure also provides a polynucleotide encoding an ACE2 signal peptide, an ACE2 extracellular domain, a linker peptide, and an anti-CD3 antibody (e.g., single-chain variable fragment; scFv). A vector comprising the polynucleotide is also provided.

A vector is a construct that is capable of delivering, and usually expressing, at least one gene or sequence of interest in a host cell. Examples of vectors include but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes. A vector may, in some embodiments, be an isolated nucleic acid that can be used to deliver a composition to the interior of the cell. The term should also be construed to include facilitate transfer of nucleic acid into cells of the non-plasmid and non-viral compounds, for example, polylysine compounds, liposomes, and the like.

Non-limiting examples of viral vectors include but are not limited to adenoviral vectors, adeno-associated virus vectors, and retroviral vectors. In some embodiments, the vector is a viral vector, for example, a lentiviral vector, retroviral vector, adenoviral vector, adeno-associated viral vector, or herpes simplex viral vector. In one embodiment, the vector is a lentiviral vector.

### IV. Pharmaceutical Compositions

Provided herein are compositions (e.g., pharmaceutical compositions), methods, kits and reagents for prevention or treatment of coronaviruses in humans and other mammals. The compositions provided herein can be used as therapeutic or prophylactic agents. They may be used in medicine to prevent and/or treat a coronavirus infection.

The term “pharmaceutical composition” refers to the combination of an active agent (e.g., polypeptide , CAR T cell, or polynucleotide described herein) with a carrier (e.g., pharmaceutically acceptable excipient), inert or active, making the composition especially suitable use in vivo or ex vivo. A “pharmaceutically acceptable excipient,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, pharmaceutical compositions comprise at least one additional active substance, such as, for example, a therapeutically-active substance, a prophylactically-active substance, or a combination of both. Compositions may be sterile, pyrogen-free or both sterile and pyrogen-free. General considerations in the formulation and/or manufacture of pharmaceutical agents, such as pharmaceutical compositions, may be found, for example, in Remington: The Science and Practice of Pharmacy 21st ed., Lippincott Williams & Wilkins, 2005 (incorporated herein by reference in its entirety).

As an example, the polypeptide, polynucleotide, or CAR T cells may be formulated or administered alone or in conjunction with one or more other components. For example, an composition may comprise other components including, but not limited to, adjuvants. In some embodiments, an immunizing composition does not include an adjuvant (they are adjuvant free).

Formulations of the compositions described herein may be prepared by any method known or hereafter developed in the art of pharmacology. In general, such preparatory methods include the step of bringing the active ingredient (e.g., polypeptide, polynucleotide, or CAR T cells) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient (polypeptide, polynucleotide, or CAR T cells).

### V. Dosing/Administration

Provided herein are compositions, methods, kits and reagents for prevention and/or treatment of a coronavirus (e.g., SARS-CoV-2) infection in humans and other mammals. The compositions can be used as therapeutic or prophylactic agents. In some embodiments, the compositions are used to provide prophylactic protection from coronavirus infections. In some embodiments, the compositions are used to treat coronavirus infections. As such, the compositions, in some embodiments, are administered to a subject in an effective amount.

An “effective amount” of a composition (e.g., comprising the polypeptides, polynucleotides, vectors, and/or CAR T cells described herein) is based, at least in part, on the target tissue, target cell type, means of administration, physical characteristics of the active ingredient(s), other components of the composition, and other determinants, such as age, body weight, height, sex and general health of the subject. Typically, an effective amount of a composition provides an induced or boosted immune response. In some embodiments, an effective amount is the amount necessary to prevent infection or reduce the severity of a coronavirus infection in the subject based on a single dose of the composition.

A subject may be any mammal, including non-human primate and human subjects. Typically, a subject is a human subject. In some embodiments, the subject is 60 years of age or older (e.g., 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 years of age or older). In some embodiments, the subject is under 18 years of age (e.g., under 17, 16, 15, 14, 13, 12, 11, 10, 9, 8, 7, 6, 5, 4, 3, 2, or 1 years of age). In some embodiments, the subject has or is suspected of having a betacoronavirus infection. In some embodiments, the subject has or is suspected of having a SARS-CoV-2 infection.

Prophylactic protection from a coronavirus can be achieved following administration of a composition (e.g., polypeptides, polynucleotides, vectors, and/or CAR T cells described herein) of the present disclosure. Compositions can be administered once, twice, three times, four times or more, as needed.

The composition (e.g., pharmaceutical composition) may be administered by any route that results in a therapeutically effective outcome. These include, but are not limited, to intradermal, intramuscular, intranasal, and/or subcutaneous administration.

Additional aspects, advantages and/or other features of example embodiments of the disclosure will become apparent in view of the detailed description provided herein, taken in conjunction with the accompanying drawings. It should be apparent to those skilled in the art that the described embodiments provided herein are merely exemplary and illustrative and not limiting. Numerous embodiments of modifications thereof are contemplated as falling within the scope of this disclosure and equivalents thereto.

### EXAMPLES

The following examples are provided to further illustrate various non-limiting embodiments and techniques of the present method, including experiments performed in developing the present method. It should be understood, however, that these examples are meant to be illustrative and do not limit the scope of the claims. As would be apparent to skilled artisans, many variations and modifications are intended to be encompassed within the spirit and scope of the disclosure.

### Example 1—Development of SARS-CoV-2 Specific Synthetic CARs Expressed in T Cells

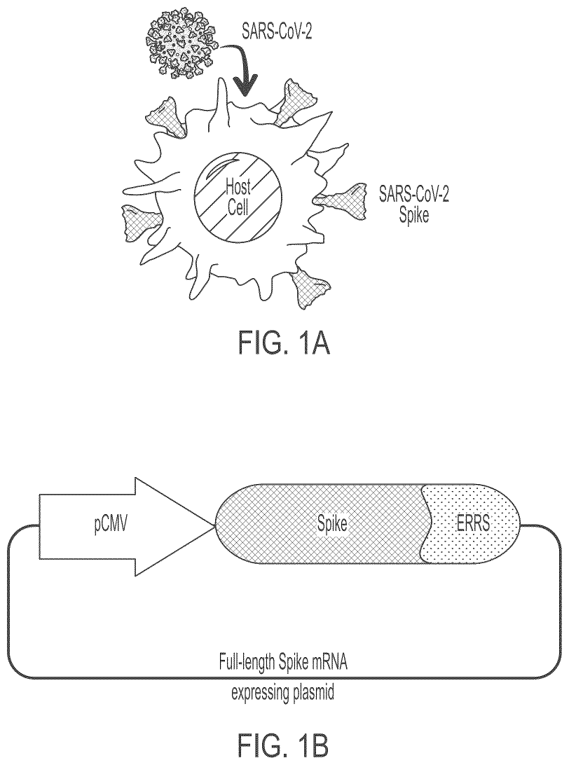

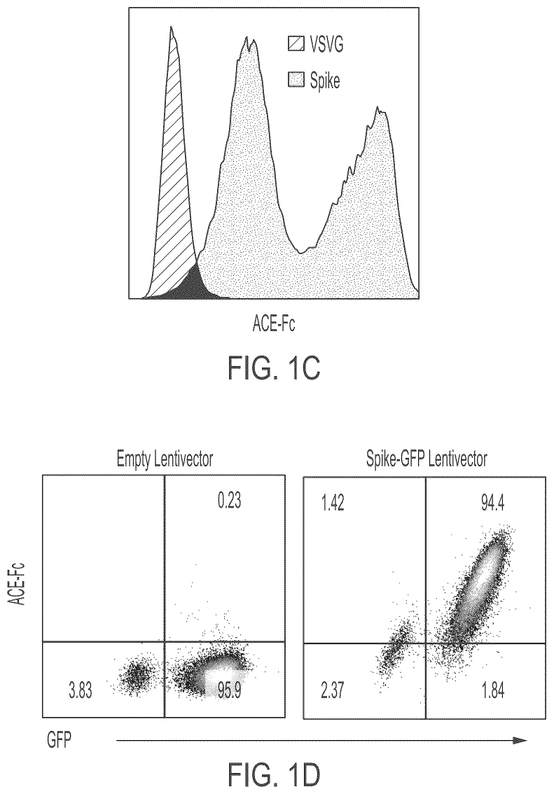

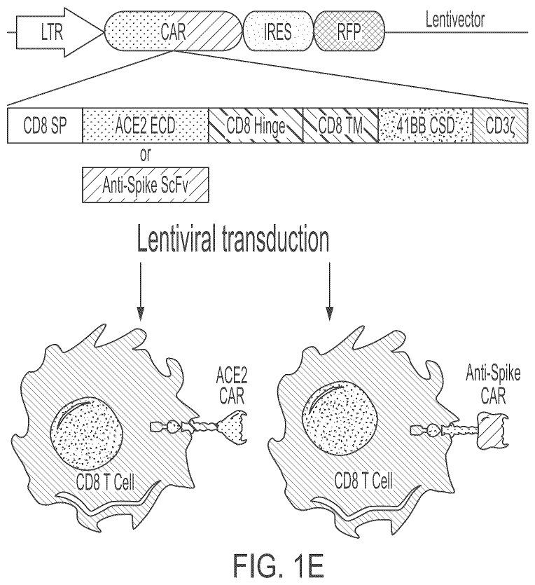

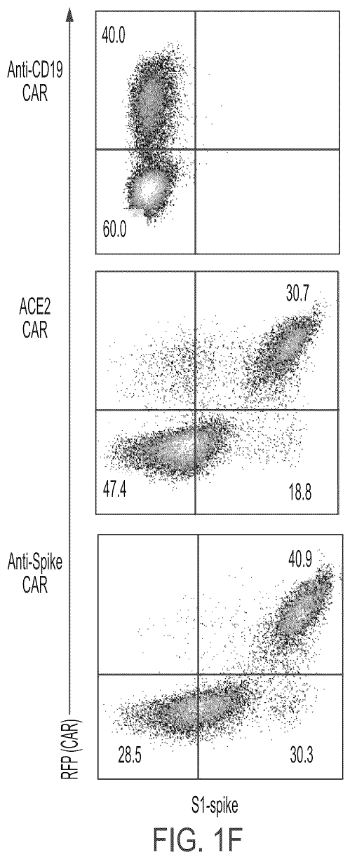

A system was developed to test whether cells that express cell surface SARS-CoV-2 Spike protein on their cell surface during the infection could be targeted (Cattin-Ortola et al., 2021) (FIG. 1A), employing effector human T cells engineered to express CAR molecules that can recognize the Spike protein on cell surface. The cells were transfected with a plasmid containing a full-length wild-type Spike protein gene under CMV promoter (FIG. 1B). 72 hours later, cells were stained with a recombinant ACE2-Fc protein followed by an anti-Fc antibody to detect surface Spike protein expression and compared to control Vesicular stomatitis Virus G (VSVG) plasmid transfected cells. 293 cells transfected with full-length Spike protein plasmid displayed cell surface Spike expression, indicating Spike protein indeed can be localized to the cell membrane despite its ERRS domain (FIG. 1C). A target 293 cell line that stably expressed both the Spike protein and Green Fluorescent Protein (GFP) as a reporter was then targeted. Further, to enhance cell surface spike protein expression as shown in previous studies (Dieterle et al., 2020; Duan et al., 2020), the ERRS domain was deleted. 72 hours after the transduction, engineered 293 cells were stained for their Spike expression, and flow cytometry analysis showed a co-expression of Spike and GFP in a high percentage of cells (FIG. 1D).

Next, lentivector constructs were designed containing ACE2 CAR or anti-Spike CAR cassettes followed by an Internal Ribosomal Entry Site (IRES) and Red Fluorescent Protein (RFP) and used to transduce human primary CD8 T cells (FIG. 1E) as previously described

(Wan et al., 2013). ACE2 CAR and anti-Spike CAR constructs comprised of CD8 alpha signal peptide, ACE2 extracellular domain (ECD) or anti-Spike ScFv, respectively, and intracellular 41BB co-stimulatory domain (CSD), and CD3 (zeta) signaling domains (FIG. 1E). An Anti-CD19 CAR-RFP lentiviral construct was also designed to be used as a control. CD8 T cells were then activated and transduced with these lentiviruses encoding the CAR constructs and expanded in IL-2 for 10-12 days. CD8 T cells engineered with ACE2 CAR and anti-Spike CAR constructs expressed these on cell surface, which also correlated with RFP reporter expression (FIG. 1F).

### Example 2—Cytotoxicity Assays with ACE2 CAR and Anti-Spike CAR Expressing T Cells

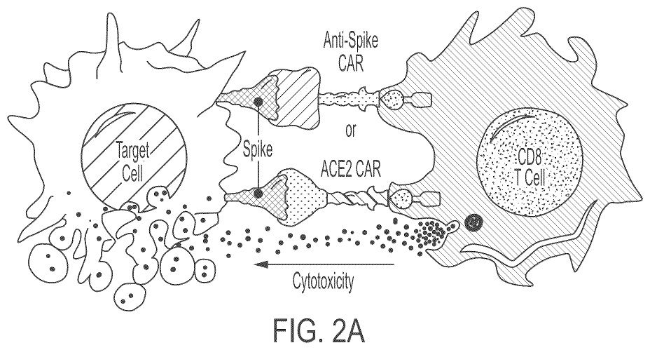

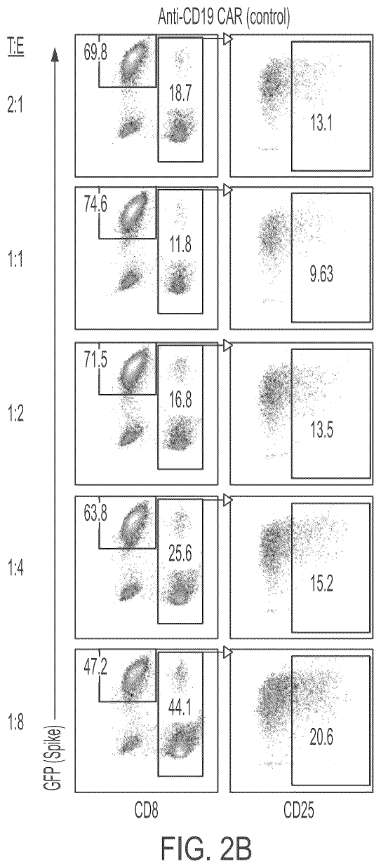

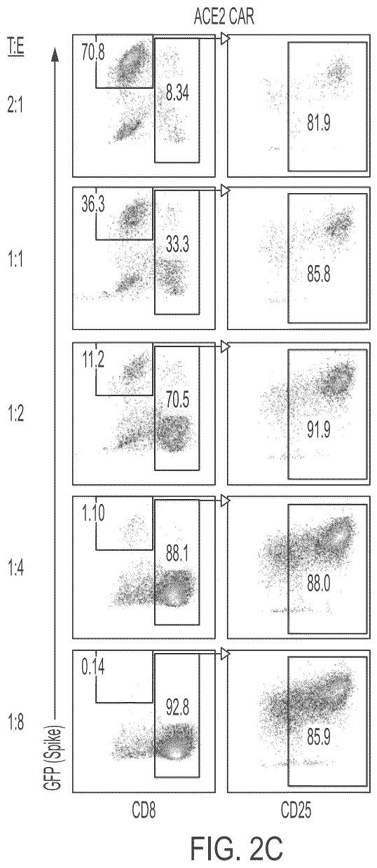

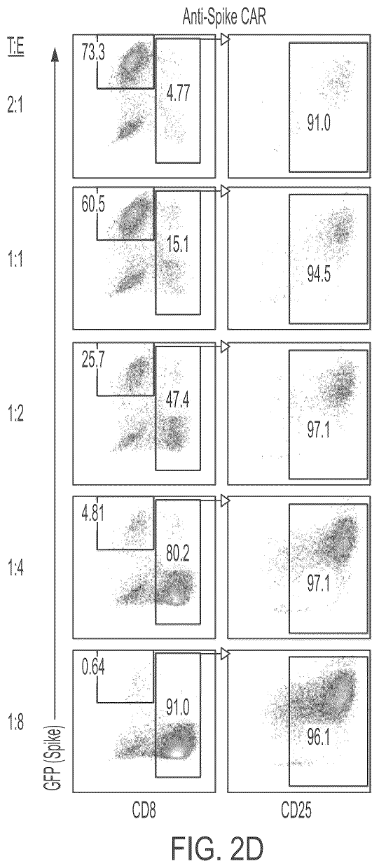

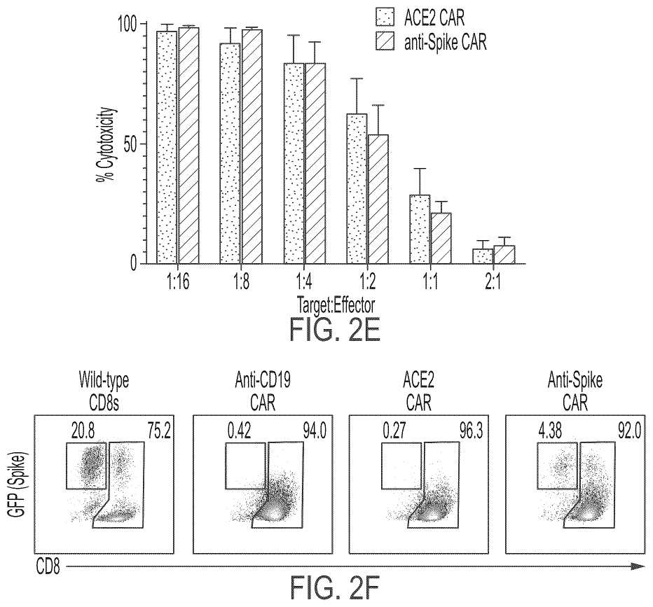

A Spike+ target cell line and effector T cells expressing CARs were then co-cultured and the cytotoxicity activity of the T cells was measured (FIG. 2A). Briefly, after the ˜2-week proliferation of CAR-T cells, the cells were co-cultured for 72 hours with Spike-expressing target cells at different effector to target ratios. The CD8+ T cells were then stained with anti-CD25 to determine their activation. Target cells were identified via GFP, which was co-expressed with Spike protein. Both ACE2 CAR and anti-Spike CAR-T cells became highly activated and killed the Spike+ 293 cells whereas control anti-CD19 CAR-T cells were neither activated nor showed any cytotoxicity (FIGS. 2B-2E).

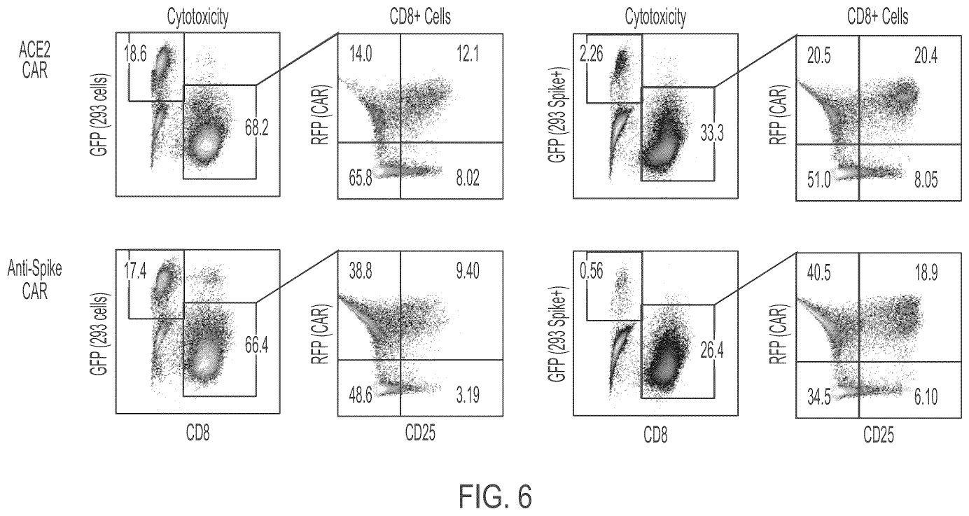

Next, ACE2 CAR and anti-Spike CAR-T cells were tested to determine whether they can kill Spike-expressing human B cell line, which was also used as positive control using anti-CD19 CAR T cells. ACE2 CAR and anti-Spike CAR-T cells killed Spike-expressing B cells as efficiently as 293 cells, indicating that different cell types infected with SARS-CoV-2 can be targeted using these novel CAR-T cells (FIG. 2F). In addition, ACE2 CAR and anti-Spike CAR-T cells did not show cytotoxicity to GFP-expressing, Spike-negative control targets and were also not activated, showing a selective Spike protein-mediated activation and killing (FIG. 6)

### Example 3—Development of Bispecific ACE2/anti-CD3 Molecules to Mobilize and Activate T Cells Against SARS-CoV-2 Spike Protein Expressing Target Cells

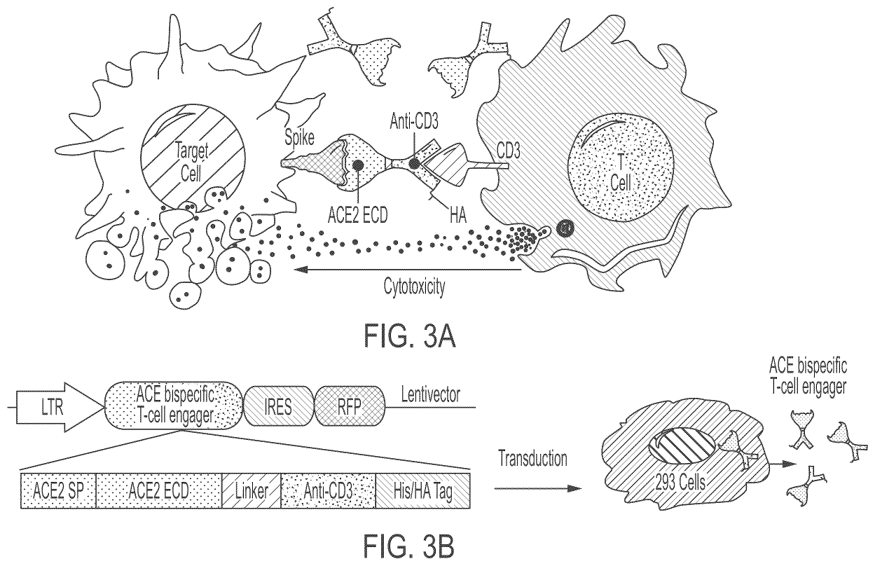

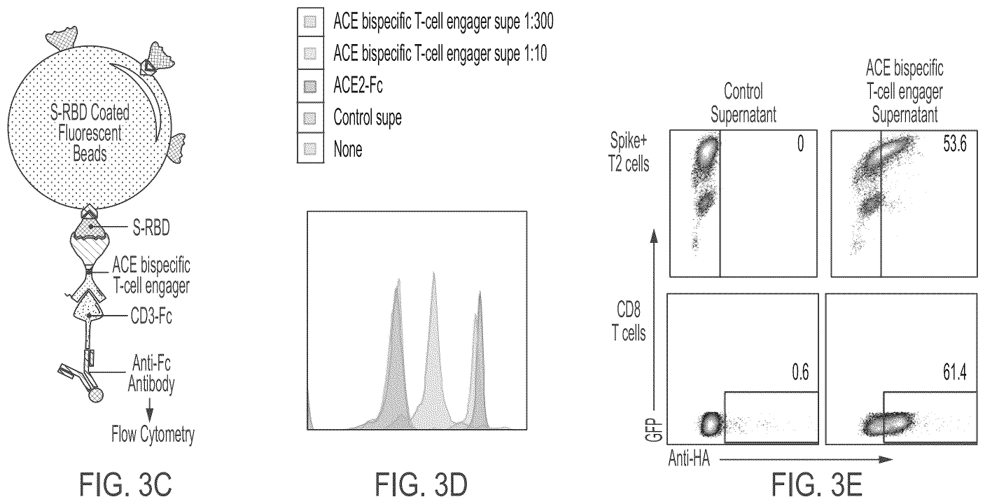

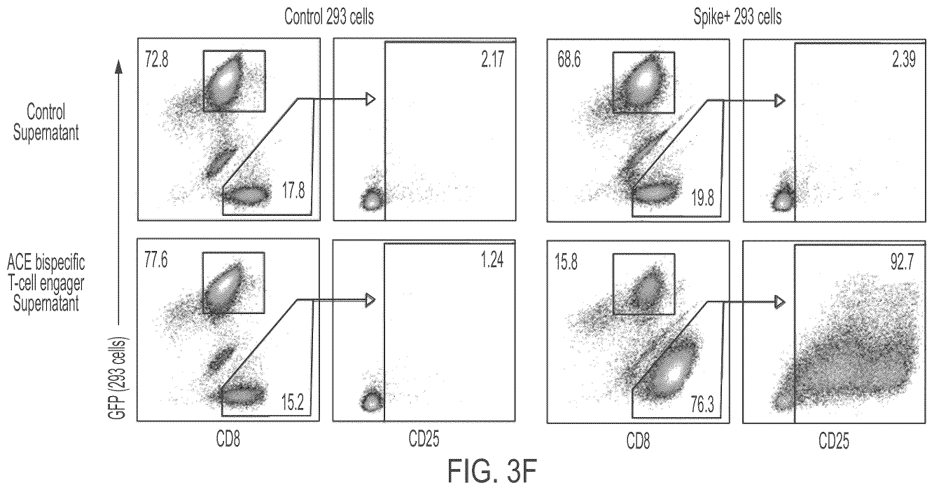

Currently, the CAR-T cell immunotherapy procedure requires a meticulous process of collecting cells from patients, engineering them in a GMP environment, re-infusion, and extensive clinical follow-up of the patients (Sterner and Sterner, 2021). As such this may not be practical for treatment of COVID-19 patients. As an alternative, bispecific ACE2/anti-CD3 molecule were engineered as T cell activators, consisting of an anti-CD3 scFv fused with the extracellular domain of ACE2 to redirect CD3 T cells to SARS-CoV-2 infected cells (FIG. 3A). The bispecific ACE2/anti-CD3 cassette consisted of ACE2 signal peptide, ACE2 extracellular domain, a linker peptide, an anti-CD3 antibody single-chain variable fragment, a His-Tag, and a Hemagglutinin (HA) Tag (FIG. 3B). Bispecific ACE2/anti-CD3 molecule was produced in suspension 293 cells as described in Example 6. The supernatant from these cells were then filtered to eliminate molecules smaller than 30 kDa, which also resulted in ˜30-fold concentration of bispecific ACE2/anti-CD3 moleculs. The supernatant of wild-type suspension 293 cells was also collected and filtered/concentrated to be used as a control.

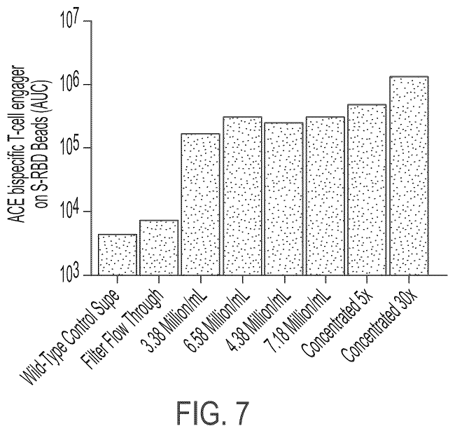

To test the correct folding of the recombinant bispecific ACE2/anti-CD3 molecule, a fluorescent bead-based bispecific ACE2/anti-CD3 molecule detection assay was developed in which the fluorescent beads were coated with Receptor Binding Domain of SARS-CoV-2 Spike protein (S-RBD) and bispecific ACE2/anti-CD3 molecules captured by S-RBD beads were detected via a recombinant CD3-Fc molecule which was then stained with an anti-Fc antibody (FIG. 3C). A recombinant ACE2-Fc molecule was used as a positive control since ACE2 part could bind to S-RBD on the surface of beads and anti-Fc antibody could recognize the Fc part of ACE2-Fc. bispecific ACE2/anti-CD3 molecule detection assay showed that detected bispecific ACE2/anti-CD3 molecule levels (1:10) were comparable to control ACE2-Fc concentration (3 μg/mL) (FIG. 3D) and correlated with bispecific ACE2/anti-CD3 molecule secreting 293 cell density (FIG. 7). It was also confirmed that bispecific ACE2/anti-CD3 molecule concentration protocol functioned as intended and increased the bispecific ACE2/anti-CD3 molecule concentration by an order of magnitude (FIG. 7).

The bispecific ACE2/anti-CD3 molecule binding was then tested on human primary CD8 T and Spike-expressing target cells. For this, bispecific ACE2/anti-CD3 molecule and wild-type supernatants were added to a B cell line (T2 cells) which was engineered to express Spike/GFP and primary human CD8 T cells. The cells combined with bispecific ACE2/anti-CD3 molecule or control supernatants were then stained for HA Tag on their surface. Spike/GFP co-expressing T2 cells and CD3 expressing primary human CD8 T cells combined with bispecific ACE2/anti-CD3 molecules were stained positive for HA Tag, suggesting Spike specific binding to ACE2 fragment and CD3 specific binding to Anti-CD3 fragment. (FIG. 3E).

A cytotoxicity assay was then performed to test the ability of bispecific ACE2/anti-CD3 molecules to trigger primary human T cell activation. Human CD8 T cells were co-cultured with Spike-expressing or control 293 cells in the presence of bispecific ACE2/anti-CD3 molecule or control supernatants. 2 days later cells were collected and stained for their CD8 and CD25 expression. GFP expressed by control and Spike lentivectors was used to identify the target cells. Indeed, resting human T cells became activated and were cytotoxic only in the presence of bispecific ACE2/anti-CD3 molecule supernatant and Spike-expressing targets, suggesting Spike-specific T-cell activation functionality of bispecific ACE2/anti-CD3 molecules (FIG. 3F).

### Example 4—Determining Function of Bispecific ACE2/Anti-CD3 Molecules on Mutated Spike Proteins

A major advantage of targeting the SARS-CoV-2 Spike protein through its receptor ACE2 is that this approach is less affected by antibody escape mutations, as mutated Spike proteins would still need to interact with ACE2. In fact, it is conceivable that variants with increased affinity to ACE2 would bind better to bispecific ACE2/anti-CD3 molecule, possibly improving its efficacy.

To test this with a bispecific ACE2/anti-CD3 molecule/Spike binding assay, 293 cells were transfected with plasmids to express 7 different mutant Spike proteins (Table 1) and the bispecific ACE2/anti-CD3 molecule binding to these Spike proteins was determined (FIG. 4A).

TABLE 1
Mutations in the Spike-Receptor Binding Domain

| Spike version | Note | Lineage |
|---|---|---|
| Wild-type Spike | Wuhan strain S-RBD | Wuhan-Hu-1 |
| E484K | In multiple variants | — |
| N501Y | Alpha variant | B.1.1.7 |
| E484K + N501Y | In multiple variants | — |
| E484K + N501Y + | Beta variant | B.1.351 |
| K417N | | |
| L452R + T478K | Delta variant | B.1.617.2 |
| L452R + T478K + | Delta plus variant | B.1.617.2.1 |
| K417N | | |

Three days after the transfection, the cells were collected and co-stained with bispecific ACE2/anti-CD3 molecule and an anti-Spike antibody and analyzed via flow cytometry. The bispecific ACE2/anti-CD3 molecule/Spike binding assay revealed that the mean fluorescent intensity of cells stained with bispecific ACE2/anti-CD3 molecule under anti-Spike antibody-stained cell population increased with some of the mutations of the Spike protein with the exception of K417N (FIG. 4B). Other studies also reported the weakening effect of K417N mutation on the affinity of Spike to ACE2 (Barton et al., 2021) (Laffeber et al., 2021). Cell cultures with these transfected 293 cells in the presence of bispecific ACE2/anti-CD3 molecule, highly efficiently activated CD8 T cells, regardless of the type of Spike protein mutations, implicating potential pan-SARS-CoV-2 effectivity of bispecific ACE2/anti-CD3 molecule approach (FIG. 4C).

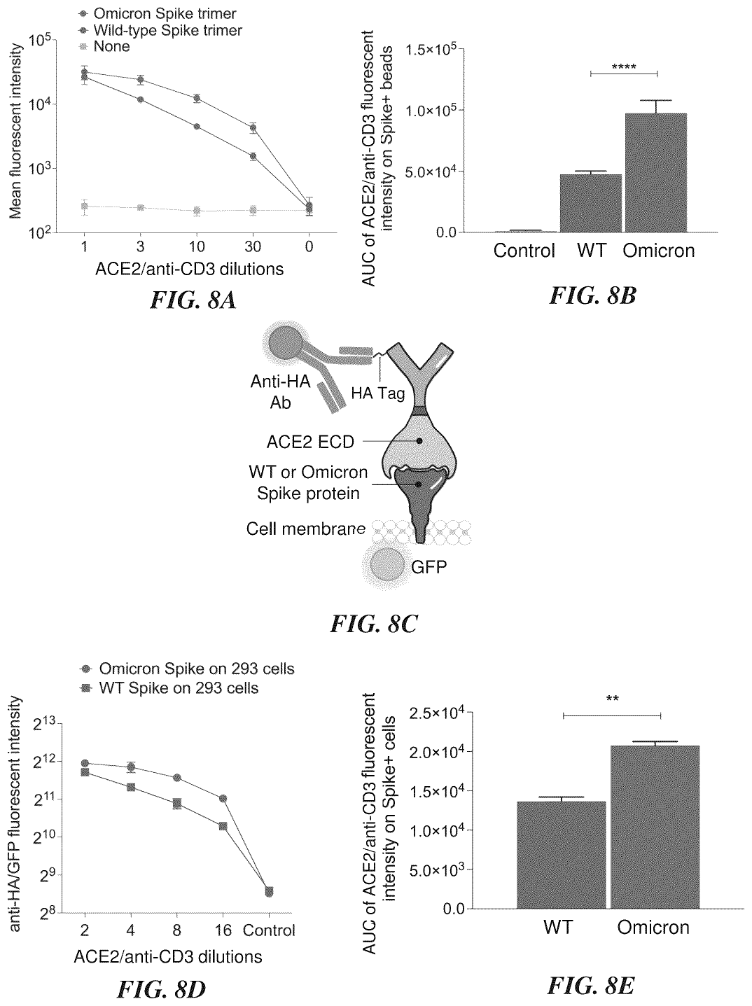

Further experiments were performed to measure the affinity of the bispecific ACE2/anti-CD3 molecule to Omicron Spike trimeric protein in comparison with wild-type Spike trimeric protein. The bead-based antibody detection strategy described in Example 3 was used. The beads were labeled with wild-type and Omicron Spike protein trimers. Labelled beads were treated with bispecific ACE2/anti-CD3 molecule and control supernatants in three-fold serial dilutions from 1 to 30 to generate titration curves. bispecific ACE2/anti-CD3 molecule treated beads were then stained with recombinant CD3-Fc protein and anti-Fc antibody and analyzed via flow cytometry. Bead-based assay using these Spike protein trimers revealed a significant increase in affinity of bispecific ACE2/anti-CD3 molecule to Omicron Spike trimer compared to wild-type (p<0.0001) (FIGS. 8A-8B).

In addition to performing affinity assay on Spike-labeled beads, 293 cells were transduced with GFP encoding lentivectors to express wild-type and Omicron variant Spike proteins and the bispecific ACE2/anti-CD3 molecule affinity to these proteins on live target cells was determined (FIG. 8C). Three days after the transduction, the cells were collected and co-stained with bispecific ACE2/anti-CD3 molecule and anti-HA antibody and analyzed via flow cytometry. The bispecific ACE2/anti-CD3 molecule/Spike protein binding assay on living cells revealed a significantly higher affinity of bispecific ACE2/anti-CD3 molecule to Omicron variant Spike protein compared to wild-type (p=0.0053), confirming the result of the bead-based affinity assay (FIGS. 8D-8E).

### Example 5—Bispecific ACE2/Anti-CD3 Molecules Neutralize Spike-Expressing Lentiviruses

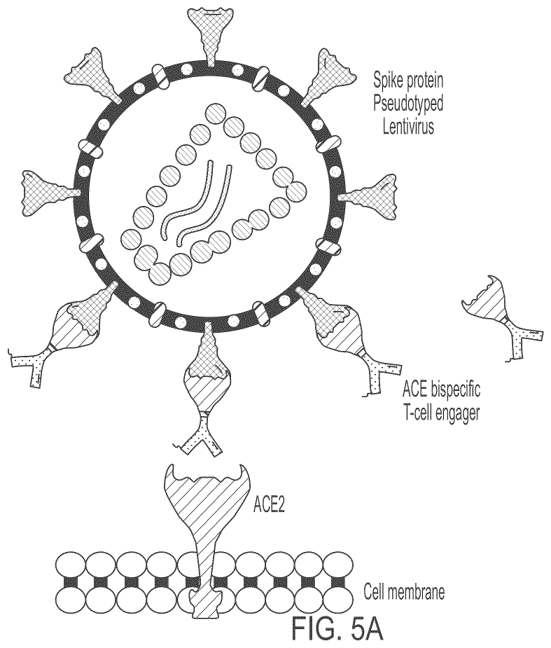

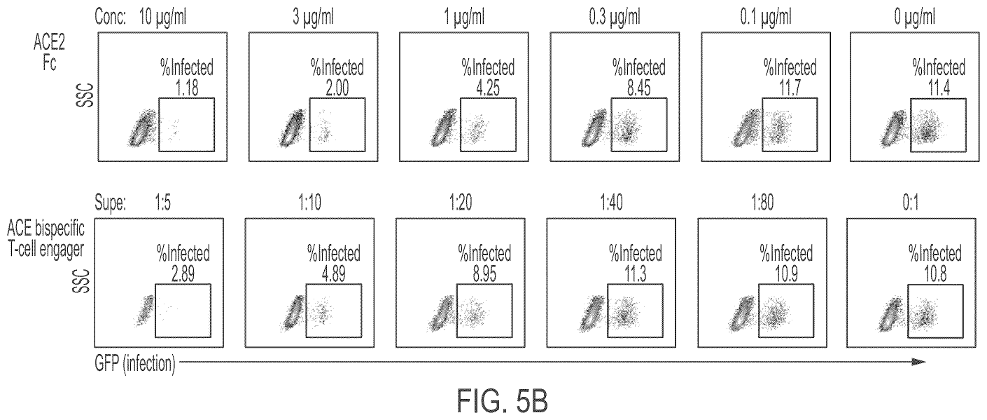

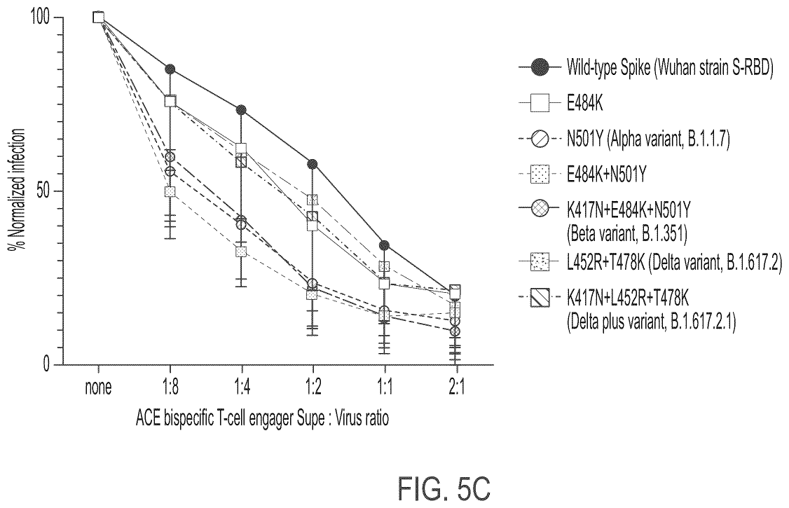

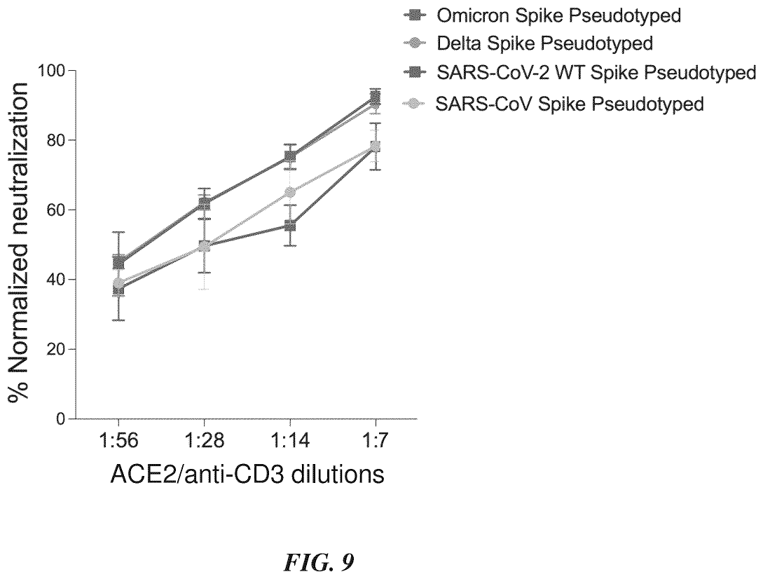

In addition to bridging infected cells to activate T cells, it was reasoned that bispecific ACE2/anti-CD3 molecule may also neutralize SARS-CoV-2 by binding to Spike proteins on the virus. To test this, a set of lentiviruses pseudotyped with mutant SARS-CoV-2 Spike proteins comprising S-RBD regions with 7 different mutations (Table 1) and exact Spike proteins of SARS-CoV, SARS-CoV-2 wild-type, Delta, and Omicron variants was generated and bispecific ACE2/anti-CD3 molecule binding to Spike was determined. Neutralization was determined by pre-culturing pseudotyped viruses with different dilutions of bispecific ACE2/anti-CD3 molecule and then adding to ACE2 expressing 293 cells as previously described (Dogan et al., 2021) (FIG. 5A). A recombinant ACE2-Fc molecule was also incubated at different concentrations with Spike pseudotyped lentivirus as a positive control. The infection levels were determined 3 days post-infection based on the GFP expression of ACE2-expres sing 293 cells. As shown in the representative experiment, ACE2-Fc and bispecific ACE2/anti-CD3 molecules neutralized the Spike pseudotyped lentivirus (FIG. 5B). Importantly, bispecific ACE2/anti-CD3 molecule was able to neutralize all of the mutant Spike encoding lentiviruses with similar efficiencies (FIG. 5C). In particular, the bispecific ACE2/anti-CD3 molecule was able to neutralize all versions of Spike encoding lentiviruses with increased efficiency against Delta and Omicron variants compared to wild-type SARS-CoV-2 pseudovirus (p=0.016, 0.0008 and 0.016 for Delta 1:28, 1:14 and 1:7 dilutions, respectively; p=0.0023 and 0.014 for Omicron 1:14 and 1:7 dilutions, respectively) (FIG. 9). These neutralization assays demonstrated that novel bispecific ACE2/anti-CD3 molecule could also function as a decoy receptor against the virus.

### Materials and Methods for Examples 1-5

### ACE2 CAR Construct

CAR constructs consisting of CD8 alpha signal peptide, extracellular domain of ACE2 molecule or single chain variable fragment (scFv) of anti-CD19 or anti-Spike protein antibodies, CD8 hinge domain, CD8 transmembrane domain, 4-1BB (CD137) intracellular domain and CD3t domain were designed with Snapgene and synthesized via Genscript. ACE2 extracellular domain, CD8a signal peptide, CD8 hinge, CD8 transmembrane domain, 4-1BB intracellular domain and CD3t domain sequences were obtained from Ensembl Gene Browser and codon optimized with SnapGene by removing the restriction enzyme recognition sites that are necessary for subsequent molecular cloning steps, while preserving the amino acid sequences. Anti-CD19 and anti-Spike scFv amino acid sequences were obtained from Addgene plasmids #79125 and #155364, respectively, reverse translated to DNA sequences and codon optimized with Snapgene 5.2.4. The constructs were then cloned into a lentiviral expression vector with a multiple cloning site separated from RFP reporter via an Internal Ribosomal Entry Site (IRES).

### Spike Protein Constructs

Human codon optimized wild-type full-length SARS-CoV-2 Spike protein sequence was synthesized by MolecularCloud (MC_0101081) and then cloned into pLP/VSVG plasmid from Thermo Fisher under CMV promoter after removing the VSVG sequence via EcoRI-EcoRI restriction digestion. 5′-CGACGGAATTCATGTTCGTCTTCCTGGTCCTG-3′ (SEQ ID NO: 8) and 5′-ACGACGGAATTCTTAACAGCAGGAGCCACAGC-3′(SEQ ID NO: 9) primers were used to generate wild-type SARS-CoV-2 Spike protein sequence without the Endoplasmic Reticulum Retention Signal (ERRS, last 19 amino acids of Spike) (Ou et al., 2020). E484K and N501Y mutated spike protein sequences without ERRS domain were obtained from VectorBuilder plasmids pRP[Exp]-CMV-human beta globin intron>S(E484K,deltaC19)/3xFLAG and pRP[Exp]-CMV-human beta globin intron>S(N501Y,deltaC19)/3xFLAG, respectively. Since wild-type Spike protein did not have the FLAG tag and efficiently incorporated into the lentiviruses, FLAG Tags in each construct were removed to have the same amino acid sequences among all Spike constructs with the exception of the necessary mutations, via PCR amplification with 5′-ACGACGGAATTCATGTTCGTTTTCCTTGTTCTGTTGC-3′(SEQ ID NO: 10) and 5′-ACGACGGAATTCTTAGCAACATGATCCGCAAGAGCA-3′ (SEQ ID NO: 11) primers and cloned into the same pLP expression plasmid. E484K+N501Y mutated, K417N+E484K+N501Y mutated (Beta variant, B.1.351, South African), L452R+T478K mutated (Delta variant, B.1.617.2) and K417N+L452R+T478K mutated (Delta plus variant, B.1.617.2.1) Spike protein sequences were built on top of E484K and N501Y mutated Spike protein sequences via overlap extension PCR using 2 new primers together with the 5′ and 3′ primers mentioned above for each single mutation insertion and cloned into the pLP expression plasmid. All mutation insertions were confirmed via Eton Bioscience DNA sequencing. Sequencing and overlap extension PCR primer sequences are available upon request.

Human codon optimized wild-type and Omicron full-length SARS-CoV-2 Spike protein sequences were synthesized by MolecularCloud (MC_0101081 and MC_0101272, respectively) and then cloned into pLP/VSVG plasmid from Thermo Fisher under CMV promoter after removing the VSVG sequence via EcoRI-EcoR restriction digestion. 5′-ACGACGGAATTCATGTTCGTCTTCCTGGTCCTG-3′ (SEQ ID NO: 12) and 5′-ACGACGGAATTCTTAACAGCAGGAGCCACAGC-3′ (SEQ ID NO: 13); and 5′-ACGACGGAATTCATGTTCGTGTTCCTGGTGCT-3′ (SEQ ID NO: 14) and 5′-ACGACGGAATTCTTAACAGCAACTGCCGCAG-3′ (SEQ ID NO: 15) primers were used to generate wild-type and Omicron SARS-CoV-2 Spike protein sequences without the Endoplasmic Reticulum Retention Signal (ERRS, last 19 amino acids of Spike), respectively. For stable wild-type Spike protein overexpression, wild-type and Omicron Spike protein sequences without ERRS domain was cloned into a lentivector with a GFP marker under LTR promoter. Human codon optimized Delta full-length SARS-CoV-2 Spike protein plasmid was synthesized by Invivogen (plv-spike-v8).

### VSVG and Spike Protein Pseudotyped Lentivirus Production

The lentiviruses pseudotyped with vesicular stomatitis virus G protein envelope were generated with HEK293T cells. Briefly, the lentivector plasmids containing the constructs were co-transfected with vesicular stomatitis virus G protein, pLP1, and pLP2 plasmids into HEK293T cells at 80-90% confluency using Lipofectamine 3000 (Invitrogen) according to the manufacturer's protocol. In the case of Spike protein pseudotyped lentiviruses, a lentivector plasmid containing GFP reporter was co-transfected with wild-type or mutated SARS-CoV-2 Spike protein plasmids in the same manner. The transfection medium was replaced with RPMI 1640 with 10% FBS 6 hours post-transfection. Viral supernatants were collected 24 to 48 hours post-transfection and filtered through a 0.45-μm syringe filter (Millipore) to remove cellular debris. A Lenti-X concentrator (Takara Bio USA) was used according to the manufacturer's protocol to concentrate the virus 10-20× and the resulting lentiviral stocks were aliquoted and stored at −80° C. To measure viral titers of VSV-G pseudotyped lentiviruses, virus preparations were serially diluted on Jurkat cells and 3 days post-infection, infected cells were measured using flow cytometry and the number of cells transduced with 1 mL of virus supernatant was calculated as infectious units per milliliter. For spike protein pseudotyped lentiviruses, to measure viral titers, virus preparations were serially diluted on ACE2 over-expressing 293 cells, which were stained for their ACE2 expressions and confirmed ˜%100 positive. 72 hours after infection, GFP positive cells were counted using flow cytometry and the number of cells transduced with virus supernatant was calculated as infectious units/per mL. Based on these titer values, primary T cells, 293 T cells and T2 cells were transduced with a multiplicity of infection (MOI) of 3-10.

### Bispecific ACE2/Anti-CD3 Molecule Design and Production

The bispecific ACE2/anti-CD3 construct consisting of ACE2 signal peptide, ACE2 extracellular domain, a linker peptide, an anti-CD3 antibody single-chain variable fragment, a His-Tag, and a Hemagglutinin (HA) Tag was designed with Snapgene and synthesized via Genscript. ACE2 signal peptide and extracellular domain sequences were obtained from Ensembl Gene Browser (Transcript ID: ENST00000252519.8). Anti-CD3 antibody single-chain variable fragment, His-Tag, and Hemagglutinin (HA) Tag sequences were obtained from Addgene plasmid #85437. The bispecific ACE2/anti-CD3 construct was cloned into an RFP marked lentivector under LTR promoter, and EXPI293F™ suspension 293 cells from ThermoFisher were transduced with the bispecific ACE2/anti-CD3 molecule expressing VSVG pseudotyped lentiviruses with multiplicity of infection of 5. The cells were then grown in EXPI293™ Expression Medium in shaking flasks for 7 days until they reached maximum viable density. Bispecific ACE2/anti-CD3 molecule containing supernatant was then collected and filtered/concentrated up to 30-fold with 30 kDa MILLIPORESIGMA™ AMICON™ Ultra Centrifugal Filter Units. Concentrated bispecific ACE2/anti-CD3 molecule and control supernatants were aliquoted and stored in 4° C.

### Engineering CAR-T Cells and Spike Expressing Target Cells

Healthy adult blood was obtained from AllCells. PBMCs were isolated using Ficoll-paque plus (GE Health care). CD8 T cells were purified using Dynal CD8 Positive Isolation Kit (from Invitrogen). CD8 T cells were >99% pure and assessed by flow cytometry staining with CD8-Pacific Blue antibody (Biolegend). Total CD8 T cells were activated using anti-CD3/CD28 coated beads (Invitrogen) at a 1:2 ratio (beads:cells) and infected with anti-CD19 CAR, anti-Spike CAR or ACE2 CAR VSVG pseudotyped lentiviral constructs with multiplicity of infection (MOI) of 5-10. The cells were then expanded in complete RPMI 1640 medium supplemented with 10% Fetal Bovine Serum (FBS, Atlanta Biologicals), 1% penicillin/streptomycin (Corning Cellgro) and 20 ng/ml of IL-2 and cultured at 37° C. and 5% CO2 supplemented incubators. Respective viruses were added 24 hours after the activation. Cells were expanded for 10-12 days and cytotoxicity assays were performed following their expansion. To generate HEK-293T cells that transiently expressed wild type and mutated spike protein (ATCC; mycoplasma-free low passage stock), the cells were transfected with Spike protein expressing pLP plasmids using Lipofectamine 3000 (Invitrogen) according to the manufacturer's protocol and stained for their spike protein expression 72 hours after the transfection as described in Staining and Flow cytometry Analysis. All engineered and wild-type HEK-293 and T2 cells were cultured in complete RPMI 1640 medium (RPMI 1640 supplemented with 10% FBS; Atlanta Biologicals, Lawrenceville, GA), 8% GlutaMAX (Life Technologies), 8% sodium pyruvate, 8% MEM vitamins, 8% MEM nonessential amino acid, and 1% penicillin/streptomycin (all from Corning Cellgro). To generate T2s and 293s with stable Spike overexpression, wild-type T2 and 293 cells were transduced with 3 MOI of Spike protein overexpressing VSVG lentivirus and proliferated. The infection levels were determined by GFP expression through Flow Cytometry analysis. For ACE2 overexpression in 293, wild-type ACE2 sequence was obtained from Ensembl Gene Browser (Transcript ID: ENST00000252519.8) and codon optimized with SnapGene by removing restriction enzyme recognition sites that are necessary for subsequent molecular cloning steps preserving the amino acid sequence, synthesized in GenScript and then cloned into a lentiviral vector. VSVG pseudotyped lentiviruses of respective constructs were generated as mentioned above and added to the cells with MOI of 3. Transduction levels were determined by ACE2 staining via Flow Cytometry 72 hours after the infection. ACE2 staining is described in Staining and flow cytometry analysis.

### Flow Cytometry Analysis

Cells were resuspended in staining buffer (PBS+2% FBS) and incubated with fluorochrome-conjugated antibodies for 30 min at 4° C. CD8 T cells were identified with CD8-Pacific Blue antibody (Biolegend). Activation of CAR CD8 T cells was determined with CD25 staining using CD25-APC antibody (Biolegend). CAR expressions of ACE2 CAR and anti-Spike

CAR and ACE2 expression of ACE2-293 cells were determined with SARS-CoV-2 S1 protein, Mouse IgG2a Fc Tag (Acro Biosystems) incubation followed with APC Goat anti-mouse IgG2a Fc Antibody (Invitrogen) staining and RFP expression. CAR expression of anti-CD19 CAR was determined with Human CD19 (20-291) Protein, Fc Tag, low endotoxin (Super affinity) (Acro) followed by a secondary staining with APC conjugated anti-human IgG Fc Antibody (Biolegend) and RFP expression. For cytotoxicity assay analysis, stably Spike protein-expressing T2 and 293 cell lines were identified with GFP marker. For Spike protein flow cytometry analysis, the cells were stained with Biotinylated Human ACE2/ACEH Protein, Fc,Avitag (Acro Biosystems), then stained with APC anti-human IgG Fc Antibody clone HP6017 (Biolegend). Samples were acquired on a BD FACSymphony A5 analyzer and data were analyzed using FlowJo (BD Biosciences).

### Cytotoxicity Assay

Following the expansion of engineered CAR-T cells for 10-12 days, the cells were analyzed for their RFP and CAR expressions. Effector to target cell ratio was calculated based on the number of CAR expressing cells. CAR expressing cells were titrated from 2:1 to 1:16 effector to target cell ratio at 2-fold dilutions while the target cell number was constant. For bispecific ACE2/anti-CD3 molecule cytotoxicity assays, resting total CD8 T cells were combined with wild-type Spike overexpressing 293 cells, empty vector transduced 293 cells, mutated Spike protein transfected 293 cells and wild-type 293 cells in a 4:1 Effector/Target cell ratio, and bispecific ACE2/anti-CD3 molecule and control supernatant were added in 1:10 supernatant/cell medium ratio. Cytotoxicity assay conditions were analyzed with Flow Cytometry at 72 hours of co-incubation and the cells were identified as described in Staining and flow cytometry analysis.

### Bispecific ACE2/Anti-CD3 Molecule Detection Assay

Supernatants from ACE2 bispecific T-cell engager secreting and wild-type suspension 293 cells were collected at several timepoints with different cell densities ranging from 3 to 7 million/mL. ACE2 bispecific T-cell engager molecules taken from 3 million/mL cell culture supernatant were concentrated 5-folds and 30-folds by using 15 mL 30 kDa MilliporeSigma™ Amicon™ Ultra Centrifugal Filter Units. To capture the ACE2 bispecific T-cell engager or ACE2-Fc molecules. The DevScreen SAv Bead kit (Essen BioScience, MI) was used. Biotinylated 2019-nCoV (COVID-19) spike protein RBD, His, Avitag, Biotinylated SARS-CoV-2 Spike Trimer, His,Avitag™ (B.1.1.529/Omicron) (MALS verified) and Biotinylated SARS-CoV-2 S protein, His.Avitag™, Super stable trimer (MALS verified) were coated to SAv Beads according to manufacturer's instructions. Confirmation of successful bead conjugation was determined by staining with anti-His Tag (Biolegend) and flow cytometry analysis. The conjugated beads were then used as capture beads in flow immunoassay where they were incubated with recombinant Human ACE2-Fc (Acro Biosystems) or ACE2 bispecific T-cell engager supernatant samples for 1 h at room temperature. Supernatant samples were assayed at a 1:1 starting dilution and three additional tenfold serial dilutions. ACE2-Fc was tested at a 30 μg/mL starting concentration and in additional five threefold serial dilutions. Detection reagent was prepared using Human CD3 epsilon Protein, Mouse IgG2a Fc Tag (Acro) and Phycoerythrin-conjugated Goat anti-Mouse IgG2a Cross-Adsorbed Secondary Antibody (Fisher) for ACE2 bispecific T-cell engager and APC anti-human IgG Fc Antibody clone HP6017 (Biolegend) for ACE2-Fc were added to the wells and incubated for another hour at room temperature. Plates were then washed twice with PBS and analyzed by flow cytometry using iQue Screener Plus (IntelliCyt, MI). Flow cytometry data were analyzed using FlowJo (BD biosciences). DevScreen SAv Beads were gated using FSC-H/SSC-H, and singlet beads gate was created using FSC-A/FSC-H. Gates for different DevScreen SAv Beads were determined based on their fluorescence signature on RL1-H/RL2-H plot (on iQue plus). PE fluorescence median, which is directly associated with each single plex beads was determined using BL2-H (on iQue plus). Geometric means of PE fluorescence in different titrations were used to generate the titration curve and the area under the curve was calculated using GraphPad Prism 9.0 software (GraphPad Software). Spike pseudotyped virus neutralization assay

Three-fold serially diluted recombinant human ACE2-Fc (Acro Biosystems) or two-fold serially diluted bispecific ACE2/anti-CD3 molecule and control supernatants were incubated with GFP-encoding SARS-CoV-2 Spike pseudotyped viruses with 0.2 multiplicity of infection (MOI) for 1 hour at 37° C. degrees. The mixtures were subsequently incubated with ACE2+ 293 cells, which were previously stained for their ACE2 expressions and confirmed ˜%100 positive before neutralization assays, for 72 h hours after which cells were collected, washed with FACS buffer (1×PBS+2% FBS) and analyzed by flow cytometry using BD FACSymphony AS analyzer. Cells that do not express GFP were used to define the boundaries between non-infected and infected cell populations. Percent infection was normalized for samples derived from cells infected with SARS-CoV-2 pseudotyped virus in the absence of ACE2-Fc or bispecific ACE2/anti-CD3 molecule.

### Statistical Analyses and Reproducibility

All statistical analyses were performed and graphs were prepared using GraphPad Prism V9 software. The numbers of repeats for each experiment were described in the associated Brief Descriptions.

All references, patents and patent applications disclosed herein are incorporated by reference with respect to the subject matter for which each is cited, which in some cases may encompass the entirety of the document. The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

1. A polypeptide comprising (a) an antibody that specifically binds to a T cell antigen and (b) a cellular receptor that binds to a coronavirus viral entry protein.

2. The polypeptide of claim 1, wherein the T cell antigen is CD3.

3. The polypeptide of claim 1, wherein the antibody is selected from an scFv, Fv, F(ab′)2, Fab, and Fab′.

4. The polypeptide of claim 3, wherein the antibody is an scFv.

5. The polypeptide of claim 4, wherein the scFv is an anti-CD3 scFv.

6. (canceled)

7. The polypeptide of claim 1, wherein the coronavirus viral entry protein is beta coronavirus Spike protein or variant thereof.

8. The polypeptide of claim 7, wherein the beta coronavirus Spike protein is a SARS-CoV-2 Spike protein.

9. The polypeptide of claim 8, wherein the SARS-CoV-2 Spike protein is a variant SARS-CoV-2 Spike protein, optionally selected from Delta (B.1.617.2 and AY lineages), Omicron (B.1.1.529 and BA lineages), Alpha (B.1.1.7 and Q lineages), Beta (B.1.351 and descendent lineages), Gamma (P.1 and descendent lineages), Epsilon (B.1.427 and B.1.429), Eta (B.1.525), Iota (B.1.526), Kappa (B.1.617.1), 1.617.3, Mu (B.1.621, B.1.621.1), and Zeta (P.2).

10. The polypeptide of claim 8, wherein the cellular receptor is angiotensin-converting enzyme 2 (ACE2) receptor or comprises an extracellular domain of human ACE2 receptor.

11. (canceled)

12. (canceled)

13. The polypeptide of claim 1, wherein the antibody is linked to the cellular receptor.

14. The polypeptide of claim 13, wherein the antibody is linked to the cellular receptor through a peptide linker.

15. (canceled)

16. The polypeptide of claim 10, further comprising an ACE2 signal peptide.

17. (canceled)

18. The polypeptide of claim 10, wherein the ACE2 receptor is a modified ACE2 receptor, relative to wild-type ACE2, that does not bind to angiotensin.

19. The polypeptide of claim 1, comprising an amino acid sequence that has at least 80 identity to the sequence of SEQ ID NO: 5 or at least 80% identity to the sequence of SEQ ID NO: 6.

20. (canceled)

21. A polynucleotide encoding the polypeptide of claim 1.

22. A polynucleotide encoding an ACE2 signal peptide, an ACE2 extracellular domain, a linker peptide, and an anti-CD3 antibody single-chain variable fragment.

23. (canceled)

24. A vector comprising the polynucleotide of claim 22.

25. (canceled)

26. (canceled)

27. A pharmaceutical composition comprising the polypeptide of claim 1 and a pharmaceutically acceptable excipient.

28. A T cell comprising a chimeric antigen receptor that comprises (a) an extracellular domain of an angiotensin-converting enzyme 2 (ACE2) receptor or (b) an anti-SARS-CoV-2 Spike scFv.

29. (canceled)

30. (canceled)

31. A method comprising administering to a subject the polypeptide of any one of the preceding claims, the vector of polypeptide of claim 1.

32. (canceled)

33. (canceled)


SEQUENCE LISTING (11 sequences)

SEQ ID NO: 1 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 2 - protein, 723 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 5 aa, synthetic construct
GGGGS

SEQ ID NO: 4 - protein, 17 aa, synthetic construct
MSSSSWLLLSLVAVTAA

SEQ ID NO: 5 - protein, 971 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 6 - protein, 988 aa, synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 7 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - DNA, 32 bp, synthetic construct
  source 1..32: mol_type = other DNA
cgacggaattcatgttcgtcttcctggtcctg

SEQ ID NO: 9 - DNA, 32 bp, synthetic construct
  source 1..32: mol_type = other DNA
acgacggaattcttaacagcaggagccacagc

SEQ ID NO: 10 - DNA, 37 bp, synthetic construct
  source 1..37: mol_type = other DNA
acgacggaattcatgttcgttttccttgttctgttgc

SEQ ID NO: 11 - DNA, 36 bp, synthetic construct
  source 1..36: mol_type = other DNA
acgacggaattcttagcaacatgatccgcaagagca